SEQUENCE LISTING (17 sequences)

SEQ ID NO: 1 - DNA, 829 bp, Mycobacterium tuberculosis
  CDS Rv0636 (201)..(629)
ccaagtgctgaaattcgagaaaccgatcgtggcgggcgacaagctgtactgcgacgtctatgtggattcggtgcgtgaggcgcacggcacccagatcatcgtgaccaagaacatcgtcaccaacgaggaaggtgacctcgtgcaggagacctatacgaccctggcgggccgtgccggcgaggatggagagggattttctgatggcgctgcgtgagttcagctcggtgaaggtcggagaccagcttccggagaagacctacccgctgacccgccaggatctggtgaactacgccggagtttcgggtgacttgaacccgattcactgggacgacgagatcgccaaggtcgtcgggctggacaccgcgatcgctcacggcatgttgacgatggggatcggcggtggctacgtcacatcctgggttggcgacccgggcgcggtcaccgagtacaacgtgcggttcactgcggtggttccggtgcccaatgacggcaagggcgccgagctggtgttcaacggtcgggtgaaatcggttgatcctgagagcaagtcggtgaccatcgcactcaccgctactaccggcggcaagaagattttcgggcgggccatcgcctcggcgaagttagcgtagtttatggcgctcaagaccgatatccgcgggatgatttggcggtacccggactacttcatcgtgggccgtgagcaatgccgcgagtttgcccgagctgtcaagtgcgaccacccggcctttttcagcgaggaagcggccgccgacctcggttacgacgcgctggttgctccgctgaccttcgtgacgatcctcgccaaata

SEQ ID NO: 2 - protein, 142 aa, Mycobacterium tuberculosis
MALREFSSVKVGDQLPEKTYPLTRQDLVNYAGVSGDLNPIHWDDEIAKVVGLDTAIAHGMLTMGIGGGYVTSWVGDPGAVTEYNVRFTAVVPVPNDGKGAELVFNGRVKSVDPESKSVTIALTATTGGKKIFGRAIASAKLA

SEQ ID NO: 3 - DNA, 877 bp, Mycobacterium tuberculosis
  CDS Rv0635 (201)..(677)
gtgcggccgaagatcactttggcatgcgaggtgtgcaagcaccgtaactacatcaccaaaaagaaccgccgcaacgacccggaccggctggagctgaagaagttctgcccgaattgcggcaaacaccaggcgcaccgcgagacgcggtaaccgccgacccgcgagcagttgctgagactgactaggtaggttctacagccgtggcgttgagcgcagacatcgttgggatgcattaccggtatcccgaccactacgaggtggagcgggagaagattcgcgagtacgccgtcgccgttcaaaacgacgacgcgtggtatttcgaggaggacggcgccgccgaactcgggtataagggcttgctggctccgttgacgtttatctgtgtgttcggctacaaggcccaggcggcgttcttcaagcatgcgaacatcgcgaccgcggaggcgcagatcgtccaggtagaccaagtgctgaaattcgagaaaccgatcgtggcgggcgacaagctgtactgcgacgtctatgtggattcggtgcgtgaggcgcacggcacccagatcatcgtgaccaagaacatcgtcaccaacgaggaaggtgacctcgtgcaggagacctatacgaccctggcgggccgtgccggcgaggatggagagggattttctgatggcgctgcgtgagttcagctcggtgaaggtcggagaccagcttccggagaagacctacccgctgacccgccaggatctggtgaactacgccggagtttcgggtgacttgaacccgattcactgggacgacgagatcgccaaggtcgtcgggctggacaccgcgatcgctcacggcatgttgacgatggggatcggcggtggctacgtcacat

SEQ ID NO: 4 - protein, 158 aa, Mycobacterium tuberculosis
VALSADIVGMHYRYPDHYEVEREKIREYAVAVQNDDAWYFEEDGAAELGYKGLLAPLTFICVFGYKAQAAFFKHANIATAEAQIVQVDQVLKFEKPIVAGDKLYCDVYVDSVREAHGTQIIVTKNIVTNEEGDLVQETYTTLAGRAGEDGEGFSDGAA

SEQ ID NO: 5 - DNA, 901 bp, Mycobacterium tuberculosis
  CDS Rv0637 (201)..(701)
gcgcggtcaccgagtacaacgtgcggttcactgcggtggttccggtgcccaatgacggcaagggcgccgagctggtgttcaacggtcgggtgaaatcggttgatcctgagagcaagtcggtgaccatcgcactcaccgctactaccggcggcaagaagattttcgggcgggccatcgcctcggcgaagttagcgtagtttatggcgctcaagaccgatatccgcgggatgatttggcggtacccggactacttcatcgtgggccgtgagcaatgccgcgagtttgcccgagctgtcaagtgcgaccacccggcctttttcagcgaggaagcggccgccgacctcggttacgacgcgctggttgctccgctgaccttcgtgacgatcctcgccaaatatgtgcaactggacttcttccgccacgtcgacgtgggcatggagacgatgcagatcgttcaggtcgaccagcggttcgtgttccacaaacccgtgctcgccggggacaagttgtgggctcggatggacatccattcggtggacgagcggttcggcgcagacatcgtcgttaccagaaacctctgcaccaacgacgacggtgagctggtcatggaggcctacaccacgctgatgggccagcagggtgatggttccgccagactcaaatgggacaaggaatccgggcaggtcatcaggaccgcgtaattagcaactggccgctgcggccatgtacactcggacctcggggttttcccaacatcggcgcgctttccgtgagttcaacgagcggagtgtcgtctccactttcggttcgcgatcaccgaacggagggcgcgcgtgtcatgtgagccccggcgtagtgggttggccagggcctggtctggtcttgcctgccaaccgcgaag

SEQ ID NO: 6 - protein, 166 aa, Mycobacterium tuberculosis
MALKTDIRGMIWRYPDYFIVGREQCREFARAVKCDHPAFFSEEAAADLGYDALVAPLTFVTILAKYVQLDFFRHVDVGMETMQIVQVDQRFVFHKPVLAGDKLWARMDIHSVDERFGADIVVTRNLCTNDDGELVMEAYTTLMGQQGDGSARLKWDKESGQVIRTA

SEQ ID NO: 7 - DNA, 1796 bp, Mycobacterium tuberculosis
gtgcggccgaagatcactttggcatgcgaggtgtgcaagcaccgtaactacatcaccaaaaagaaccgccgcaacgacccggaccggctggagctgaagaagttctgcccgaattgcggcaaacaccaggcgcaccgcgagacgcggtaaccgccgacccgcgagcagttgctgagactgactaggtaggttctacagccgtggcgttgagcgcagacatcgttgggatgcattaccggtatcccgaccactacgaggtggagcgggagaagattcgcgagtacgccgtcgccgttcaaaacgacgacgcgtggtatttcgaggaggacggcgccgccgaactcgggtataagggcttgctggctccgttgacgtttatctgtgtgttcggctacaaggcccaggcggcgttcttcaagcatgcgaacatcgcgaccgcggaggcgcagatcgtccaggtagaccaagtgctgaaattcgagaaaccgatcgtggcgggcgacaagctgtactgcgacgtctatgtggattcggtgcgtgaggcgcacggcacccagatcatcgtgaccaagaacatcgtcaccaacgaggaaggtgacctcgtgcaggagacctatacgaccctggcgggccgtgccggcgaggatggagagggattttctgatggcgctgcgtgagttcagctcggtgaaggtcggagaccagcttccggagaagacctacccgctgacccgccaggatctggtgaactacgccggagtttcgggtgacttgaacccgattcactgggacgacgagatcgccaaggtcgtcgggctggacaccgcgatcgctcacggcatgttgacgatggggatcggcggtggctacgtcacatcctgggttggcgacccgggcgcggtcaccgagtacaacgtgcggttcactgcggtggttccggtgcccaatgacggcaagggcgccgagctggtgttcaacggtcgggtgaaatcggttgatcctgagagcaagtcggtgaccatcgcactcaccgctactaccggcggcaagaagattttcgggcgggccatcgcctcggcgaagttagcgtagtttatggcgctcaagaccgatatccgcgggatgatttggcggtacccggactacttcatcgtgggccgtgagcaatgccgcgagtttgcccgagctgtcaagtgcgaccacccggcctttttcagcgaggaagcggccgccgacctcggttacgacgcgctggttgctccgctgaccttcgtgacgatcctcgccaaatatgtgcaactggacttcttccgccacgtcgacgtgggcatggagacgatgcagatcgttcaggtcgaccagcggttcgtgttccacaaacccgtgctcgccggggacaagttgtgggctcggatggacatccattcggtggacgagcggttcggcgcagacatcgtcgttaccagaaacctctgcaccaacgacgacggtgagctggtcatggaggcctacaccacgctgatgggccagcagggtgatggttccgccagactcaaatgggacaaggaatccgggcaggtcatcaggaccgcgtaattagcaactggccgctgcggccatgtacactcggacctcggggttttcccaacatcggcgcgctttccgtgagttcaacgagcggagtgtcgtctccactttcggttcgcgatcaccgaacggagggcgcgcgtgtcatgtgagccccggcgtagtgggttggccagggcctggtctggtcttgcctgccaaccgcgaag

SEQ ID NO: 8 - DNA, 27 bp, Artificial
  Primer
gattttctgatggcgctgcgtgagttc

SEQ ID NO: 9 - DNA, 22 bp, Artificial
  Primer
cggtcttgagcgccataaacta

SEQ ID NO: 10 - DNA, 49 bp, Artificial
  Primer
atggctcatcatcatcatcatcatggtgcgctgcgtgagttcagctcgg

SEQ ID NO: 11 - DNA, 46 bp, Artificial
  Primer
atggctcatcatcatcatcatcatggtgcgttgagcgcagacatcg

SEQ ID NO: 12 - DNA, 20 bp, Artificial
  Primer
cagttgctaattacgcggtc

SEQ ID NO: 13 - protein, 166 aa, Mycobacterium tuberculosis
MAHHHHHHGALSADIVGMHYRYPDHYEVEREKIREYAVAVQNDDAWYFEEDGAAELGYKGLLAPLTFICVFGYKAQAAFFKHANIATAEAQIVQVDQVLKFEKPIVAGDKLYCDVYVDSVREAHGTQIIVTKNIVTNEEGDLVQETYTTLAGRAGEDGEGFSDGAA

SEQ ID NO: 14 - protein, 150 aa, Mycobacterium tuberculosis
MAHHHHHHGALREFSSVKVGDQLPEKTYPLTRQDLVNYAGVSGDLNPIHWDDEIAKVVGLDTAIAHGMLTMGIGGGYVTSWVGDPGAVTEYNVRFTAVVPVPNDGKGAELVFNGRVKSVDPESKSVTIALTATTGGKKIFGRAIASAKLA

SEQ ID NO: 15 - protein, 18 aa, Mycobacterium tuberculosis
  MISC_FEATURE (1)..(1): Xaa = Y or F
XXXXXGDXNPXHXXXXXX

SEQ ID NO: 16 - protein, 18 aa, Mycobacterium tuberculosis
YAGVSGDLNPIHWDDEIA

SEQ ID NO: 17 - protein, 23 aa, Mycobacterium tuberculosis
  MISC_FEATURE (2)..(2): Xaa = any amino acid
FXXGHXPXXXXXPGVXXXEXXAQ

(19) United States
(12) Patent Application Publication
(10) Pub. No.: US 2010/0143940 A1
(43) Pub. Date: Jun. 10, 2010

(54) (3R)-HYDROXYACYL-ACP DEHYDRATASE ENZYMES USED IN THE BIOSYNTHESIS OF MYCOLIC ACIDS AND USE OF SAME FOR SCREENING ANTIBIOTICS

(75) Inventors: Annaik Quemard, Montgiscard (FR); Mamadou Daffe, Toulouse (FR); Emmanuelle Sacco, Ramonville St Agne (FR); Thomas Alwyn Jones, Uppsala (SE); Adrian Suarez Covarrubias, Uppsala (SE); Kristina Backbro, Orsundsbro (SE)

Correspondence Address: OBLON, SPIVAK, MCCLELLAND MAIER & NEUSTADT, L.L.P., 1940 DUKE STREET, ALEXANDRIA, VA, 22314, US

(73) Assignee: CENTRE NATIONAL DE LA RECHERCHE SCIENTIFIQUE, Paris (FR)

(21) Appl. No.: 12/526,452

(22) PCT Filed: Feb. 8, 2008
(86) PCT No.: PCT/FR2008/000162

§ 371 Date: Feb. 5, 2010

(30) Foreign Application Priority Data
Feb. 9, 2007 (FR) 0700930

(51) Int. Cl. G01N 33/573; C12Q 1/34; C12N 9/14; C12N 1/20; C12N 15/63; C07H 21/04

(52) U.S. Cl. 435/7.4; 435/18; 435/69.1; 435/195; 435/252.3; 435/320.1; 536/23.2

(57) ABSTRACT

The invention relates to (3R)-hydroxyacyl-ACP dehydratase enzymes involved in the biosynthesis of mycolic acids, and to the use of same for screening antibiotics, medicaments that can be used to treat infections in humans or in animals, caused by Corynebacterineae, more specifically infections caused by pathogenic mycobacteria (Mycobacterium tuberculosis, M. africanum, M. leprae, M. ulcerans, M. microti, M. bovis, M. abscissus, M. avium, M. fortuitum, M. kansasii . . . ), and infections caused by other genera such as Nocardia, Rhodococcus, Gordona . . .

The present invention relates to novel enzymes involved in the biosynthesis of mycolic acids and to the use thereof for screening antibiotics, medicaments that can be used to treat infections in humans or in animals, caused by Corynebacterineae, and more particularly infections caused by pathogenic mycobacteria (Mycobacterium tuberculosis, M. africanum, M. leprae, M. ulcerans, M. microti, M. bovis, M. abscissus, M. avium, M. fortuitum, M. kansasii, etc.) and also infections caused by other genera such as Nocardia, Rhodococcus, Gordona, etc.

Over the past fifteen or so years, a new upsurge in tuberculosis (the agent of which is M. tuberculosis) has been observed, in particular in industrialized countries, due both to the multiplication of cases of tuberculosis/HIV coinfection and to the appearance of tubercular bacillus strains multiresistant to antibiotics. Thus, the design of new antitubercular medicaments has become a priority. The identification of pharmacological targets is therefore necessary for the development of new medicaments.

Mycobacterial mycolic acids, also known as eumycolates, are α-alkylated and β-hydroxylated very long-chain (C₆₀-C₉₀) fatty acids present in the form of esters in the wall of bacteria of a particular phylogenetic line of actinomycetes, the suborder Corynebacterineae, also called “mycolata”, comprising, inter alia, the bacterial genera: Mycobacterium, Corynebacterium, Rhodococcus, Nocardia, Gordona and Tsukamurella. The mycolic acids are thus specific for the envelope of mycobacteria and of related bacteria (Brennan & Nikaido, 1995); a distinction can be made between mycobacterial mycolic acids and the mycolic acids of related genera, which have shorter chain lengths (Barry et al. 1998). These compounds are important to the architecture and the permeability of the bacterial envelope for which they represent a hydrophobic diffusion barrier (Brennan & Nikaido, 1995). In addition, they have an essential role in the survival of mycobacteria, and they are involved in the virulence and the persistence of the tubercular bacillus in the infected host (Dubnau et al., 2000; Glickman et al., 2000).

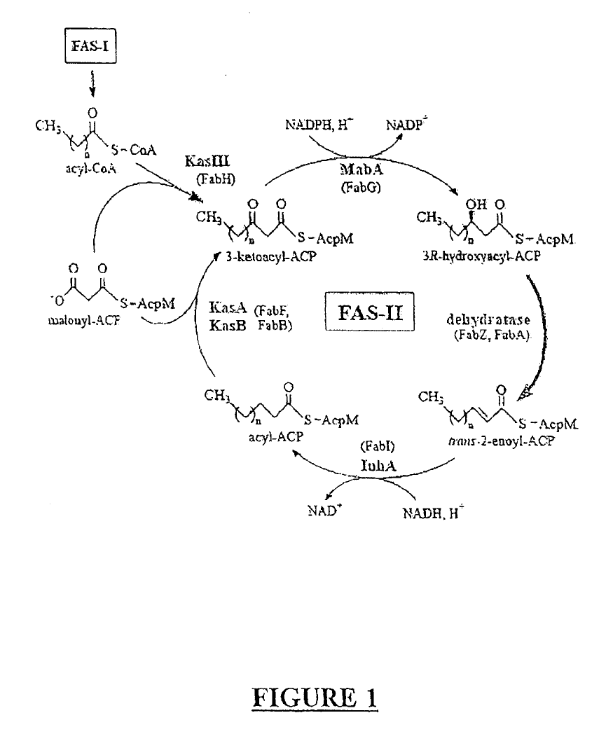

Among the mycolata are major pathogens, in particular the mycobacteria Mycobacterium tuberculosis, which is the tuberculosis agent, and Mycobacterium leprae, which is the leprosy agent. More specifically, the Mycobacterium genus comprises several species classified in 3 groups as a function of their risk of infection with respect to humans. Several species are found in the strict pathogen group, including the leprosy bacillus M. leprae and also M. tuberculosis which belongs to the subgroup known as “M. tuberculosis complex”. This subgroup is made up of 4 conventional members: M. tuberculosis, M. bovis, M. microti and M. africanum, and of the 3 recently defined members M. canetti, M. pinnipedii and M. caprae (Aranaz et al., 2003; Cousins et al., 2003; van Soolingen et al., 1997). The second group corresponds to the human opportunistic pathogens (such as M. avium, M. fortuitum, M. kansasii, etc.) which have been isolated from certain animals and in the environment. These mycobacteria are responsible for pathologies in humans (mycobacterioses), in particular in individuals with a weak immune system (for example, individuals suffering from AIDS). Finally, the third group corresponds to the nonpathogenic saprophytic mycobacteria (M. smegmatis, M. phlei, M. gastri, etc.) present in the environment. Among the antitubercular medicaments are those which interfere with mycobacterial envelope biosynthesis, such as isoniazid, ethionamide and ethambutol (WEBB et al., Molecular Biology and Virulence I: 287-307 (eds. Ratledge, C. & Dale, J.) (Blackwell Science Ltd, Oxford), 1999). The principle target of isoniazid, specifically antitubercular antibiotic, is any enzyme of mycolic acid metabolism: it inhibits the activity of the InhA protein, which is part of the FAS-II enzymatic complex or system, the function of which is to produce, by successive cycles of elongation, long-chain fatty acids (acyl-ACP up to C₃₂ in vitro, or up to C₆₀ in vivo), which are precursors of mycolic acids, using malonyl-ACP as elongation unit. InhA, a 2-trans-enoyl-ACP reductase, catalyzes the 4th step of an elongation cycle which comprises four steps (FIG. 1). The FAS-II system is present in plants, bacteria (for example, Rhodococcus, Nocardia and Mycobacterium), parasites (for example, Plasmodium) and in mitochondria.

More specifically, the initiation of the elongation system of the mycobacterial FAS-II system is carried out by a β-ketoacyl-ACP synthase III (mtFabH or KasIII), which probably makes the connection between the two systems FAS-I and FAS-II by catalyzing the condensation of the acyl-CoA derived from FAS-I with the elongation unit, malonyl-ACP (Choi et al., 2000) (FIG. 1). The FAS-II system, for its part, uses the following proteins: β-ketoacyl-ACP reductase MabA (2nd step), enoyl-ACP reductase InhA (4th step), and the β-ketoacyl-ACP synthases KasA and KasB (1st step) (Banerjee et al., 1998; Kremer et al., 2001; Kremer et al., 2002; Marrakchi et al., 2000; Quemard et al., 1995; Schaeffer et al., 2001b) (FIG. 1). The target of isoniazid is the InhA enzyme; isoniazid is a prodrug which forms, with the coenzyme of InhA, NADH, an inhibitory adduct. Initially, MabA was chosen as target and the screening of MabA-inhibiting molecules (international application WO 03/082911) was proposed.

However, there exists a real need to find inhibitors of other enzymes of this cycle, in order to broaden the panopy of available medicaments, given the frequency of appearance of antibiotic resistances. Furthermore, multiplying the targets increases the chances of designing pharmacologically active molecules which may actually be used clinically.

Among the enzymes of the mycobacterial FAS-II system, the (3R)-hydroxyacyl-ACP dehydratase enzyme, which is involved in the third step of the cycle and catalyzes the conversion of (3R)-hydroxyacyl-ACP into 2-trans-enoyl-ACP, has not yet been identified. The hydroxyacyl dehydratase enzymes or domains, as a whole, are difficult to identify owing to the lack of similarity between the sequences. This was, for example, the case of the dehydratase domain of FAS-I of M. bovis BCG and of the (3R)-hydroxyacyl-ACP dehydratase of the mitochondrial FAS-II system of Saccharomyces cerevisiae (Fernandes & Kolattukudy, 1996; Kastaniotis et al., 2004). A (3R)-hydroxyacyl-ACP dehydratase activity has been observed in bacteria (including Streptococcus, Staphylococcus, E. coli and Haemophilus influenzae), and corresponds to the FabZ (dehydratase) and FabA (dehydratase-isomerase) enzymes (patent U.S. Pat. No. 6,951,729).

Research for proteins carrying the FabA/FabZ consensus motif (‘F-x(1,2)-G-H-[FI]-P-x(5)-P-G-V-x(3)-E-[AGS]-[LM]-A-Q’) (SEQ ID No. 17) in the Mycobacterium genus using the ScanProsite software (www.expasy.ch/tools/scanProsite) has not made it possible to identify candidate (3R)-hydroxyacyl dehydratase proteins in mycobacteria. These results have made it possible to conclude that no protein of FabZ or FabA type is present in the mycobacterial species available through the ScanProsite software.

This would suggest that the (R)-specific dehydratase/hydratase proteins of mycobacteria must have a catalytic motif different than the common FabA/FabZ motif.

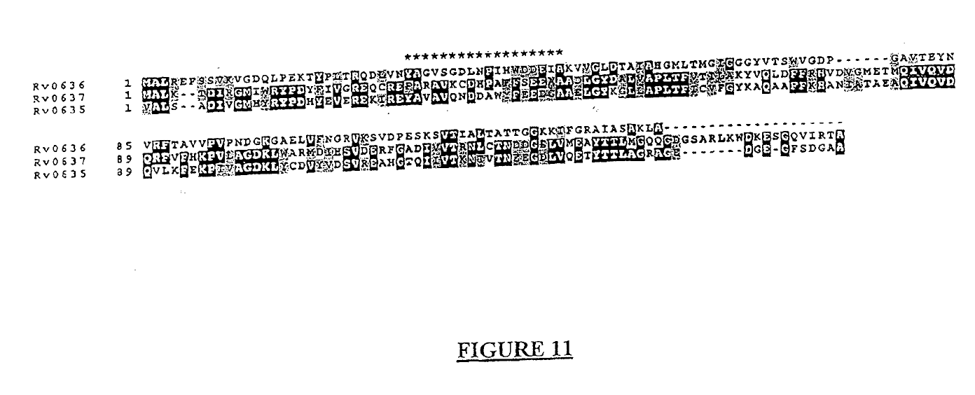

The inventors have now determined the enzymes involved in the third step of the fatty acid elongation cycle of the mycobacterial FAS-II system and the advantage thereof as a target for screening medicaments and in particular antibiotics, active on microorganisms (bacteria, parasites, for example) containing a FAS-II system, in which the third step of the elongation cycle is catalyzed by a dehydratase containing a hydratase 2 motif (FIG. 11) or a catalytic motif derived from the hydratase 2 motif and constituted at least of the basic motif D-x(4)-H, in which x(4) represents four amino acids, these amino acids being any amino acid, or of a similar basic motif.

The term “similar basic motif” is intended to mean the basic motif D-x(4)-H, in which the aspartic acid (D) is replaced with an amino acid chosen from the group constituted of asparagine (N), glutamic acid (E) and glutamine (Q), and/or the histidine (H) is replaced with a basic amino acid such as arginine (R) or lysine (L).

Molecules having an inhibitory capacity on the expression or the activity of (3R)-hydroxyacyl-ACP dehydratases of the mycobacterial FAS-II system may also potentially inhibit other essential metabolisms involving proteins of hydratase 2 type, in a given microorganism.

The inventors have found that the enzymes responsible for the third step (dehydration) in the FAS-II system of M. tuberculosis H37Rv correspond to proteins comprising protein subunits including Rv0635 (SEQ ID No. 4), Rv0636 (SEQ ID No. 2) or Rv0637 (SEQ ID No. 6) of M. tuberculosis H37Rv. Various enzymes containing Rv0636 are ACP-dependent and have a specificity for long-chain substrates.

Consequently, a subject of the present invention is a purified and isolated enzyme involved in the FAS-II system, preferably the mycobacterial FAS-II system, and having the following characteristics:
a) it is constituted of a dimer or a multimer selected from the group constituted of:
(i) homomultimers comprising at least three identical proteins comprising a hydratase 2 motif [YF]-x(1,2)-[LIVG]-[STGC]-G-D-x-N—P-[LIV]-H-x(5)-[AS] (SEQ ID No. 15) (in which x(n) represents n amino acids, these amino acids being any amino acid, and the amino acids between square brackets representing alternatives) or a catalytic motif derived from the hydratase 2 motif and constituted at least of the basic motif D-x(4)-H, in which x(4) represents 4 amino acids, these amino acids being any amino acid, or a motif similar to the basic motif,
(ii) heterodimers and heteromultimers constituted of at least two different proteins as defined in (i), or of one or more proteins as defined in (i) and of one or more proteins not carrying a hydratase 2 motif, but the three-dimensional structure of which exhibits a hotdog fold, preferably of a protein as defined in (i) and of a protein not carrying a hydratase 2 motif, but the three-dimensional structure of which exhibits a hotdog fold; and
b) it catalyzes the dehydration of a (3R)-hydroxyacyl substrate so as to give 2-trans-enoyl and/or the hydration of a 2-trans-enoyl substrate so as to give (3R)-hydroxyacyl, in accordance with the following scheme:

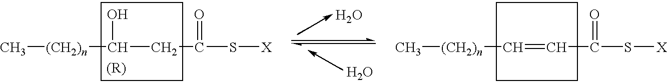

in which X represents ACP and n≧0, preferably n≧4, or CoA and n≧0, preferably n≧8.

The protein not carrying a hydratase 2 motif (i.e. without a catalytic site) is, for example, a long-acyl-chain-binding protein.

The term “multimer” signifies at least three proteins.

The methods for identifying a protein, the three-dimensional structure of which exhibits a hotdog fold, are well known. By way of example, proteins, the three-dimensional structure of which exhibits a hotdog fold, have been described by Leesong et al., 1996 and Hisano et al., 2003.

According to one advantageous embodiment of the invention, the protein as defined in (i) is selected from the group constituted of the Rv0636 protein (SEQ ID No. 2) comprising a hydratase 2 motif Y-A-G-V-S-G-D-L-N—P—I—H—W-D-D-E-I-A (SEQ ID No. 16) and a protein which has at least 51% identity or at least 72% similarity, preferably at least 85% identity or at least 90% similarity, with the Rv0636 protein SEQ ID No. 2 of M. tuberculosis H37Rv.

According to another advantageous embodiment of the invention, the protein as defined in (ii), which does not comprise a hydratase 2 motif, is selected from the group constituted of:
the Rv0635 protein (SEQ ID No. 4) or a protein which has at least 53% identity or at least 67% similarity, preferably at least 69% identity or at least 82% similarity, with the Rv0635 protein of sequence SEQ ID No. 4 of M. tuberculosis H37Rv, and
the Rv0637 protein (SEQ ID No. 6) or a protein which has at least 44% identity or at least 61% similarity, preferably at least 59% identity or at least 76% similarity, with the Rv0637 protein of sequence SEQ ID No. 6 of M. tuberculosis H37Rv.

According to another advantageous embodiment of the invention, the dimer or multimer is selected from the group constituted of:
a homomultimer of Rv0636 (SEQ ID No. 2) or of a protein which has at least 51% identity or at least 72% similarity, preferably at least 85% identity or at least 90% similarity, over its entire sequence, with the Rv0636 protein of M. tuberculosis,
a heterodimer or a heteromultimer constituted (i) of the Rv0636 protein (SEQ ID No. 2) or of a protein which has at least 51% identity or at least 72% similarity, preferably at least 85% identity or at least 90% similarity, with the Rv0636 protein of sequence SEQ ID No. 2 of M. tuberculosis H37Rv and (ii) of the Rv0635 protein (SEQ ID No. 4) or of a protein which has at least 53% identity or at least 67% similarity, preferably at least 69% identity or at least 82% similarity, with the Rv0635 protein of sequence SEQ ID No. 4 of M. tuberculosis H37Rv, and
a heterodimer or a heteromultimer constituted (i) of the Rv0636 protein (SEQ ID No. 2) or of a protein which has at least 51% identity or at least 72% similarity, preferably at least 85% identity or at least 90% similarity, with the Rv0636 protein of sequence SEQ ID No. 2 of M. tuberculosis H37Rv and (ii) of the Rv0637 protein (SEQ ID No. 6) or of a protein which has at least 44% identity or at least 61% similarity, preferably at least 59% identity or at least 76% similarity, with the Rv0637 protein of sequence SEQ ID No. 6 of M. tuberculosis H37Rv.

The enzymes in accordance with the invention are thus involved in the third step of dehydration of the elongation cycle of the FAS-II system, and in particular the mycobacterial FAS-II system, as (3R)-hydroxyacyl dehydratase (FIG. 1).

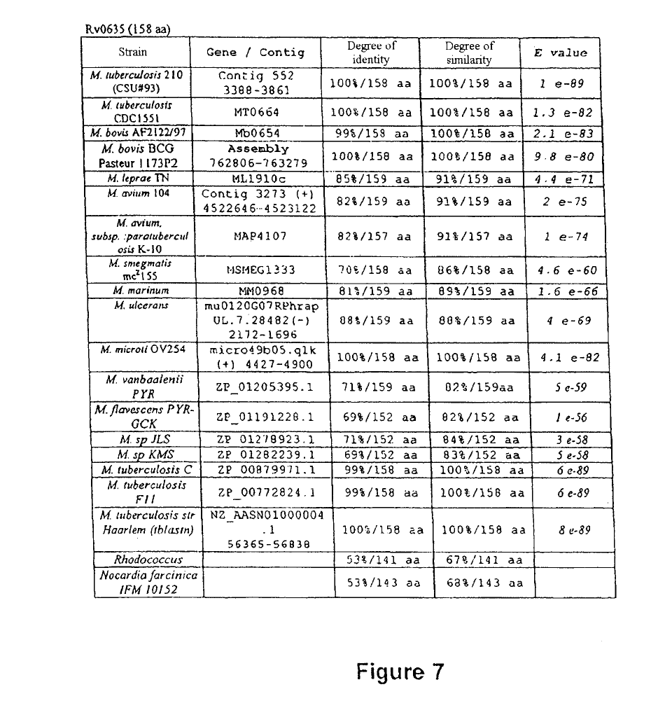

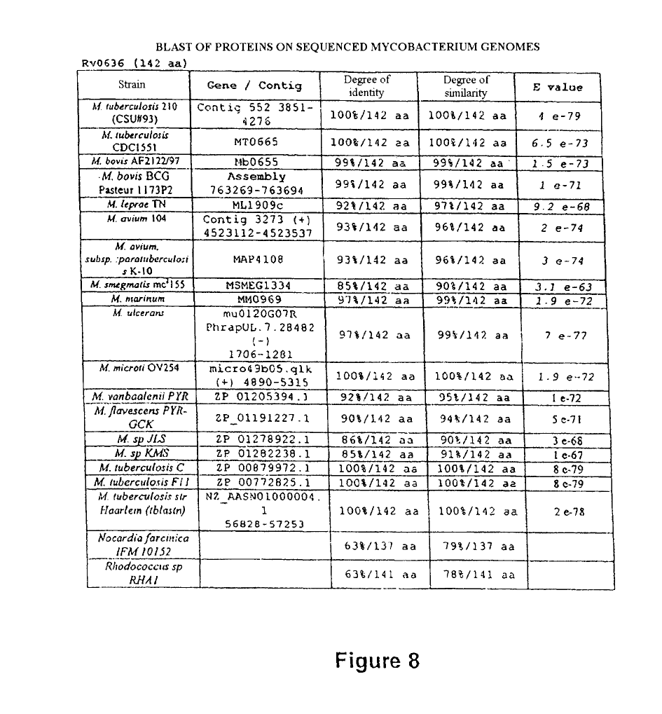

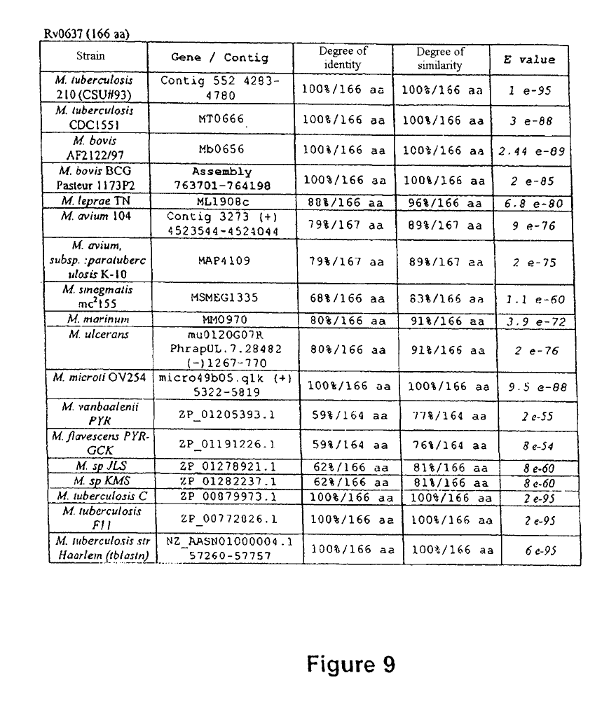

The BLAST searches have made it possible to show that the three proteins are very conserved among mycobacteria (FIGS. 7-9):
Rv0635: 70-100% identity and 86-100% similarity in M. tuberculosis and M. avium, M. smegmatis, M. bovis and M. leprae; 53% identity and 67% similarity in Nocardia and Rhodococcus.
Rv0636: 85-100% identity and 90-100% similarity in M. tuberculosis, M. bovis, M. leprae, M. avium and M. smegmatis; 51-63% identity and 72-78% similarity in Nocardia and Rhodococcus.
Rv0637: 68-100% identity and 83-100% similarity in M. tuberculosis, M. avium, M. smegmatis, M. bovis and M. leprae.

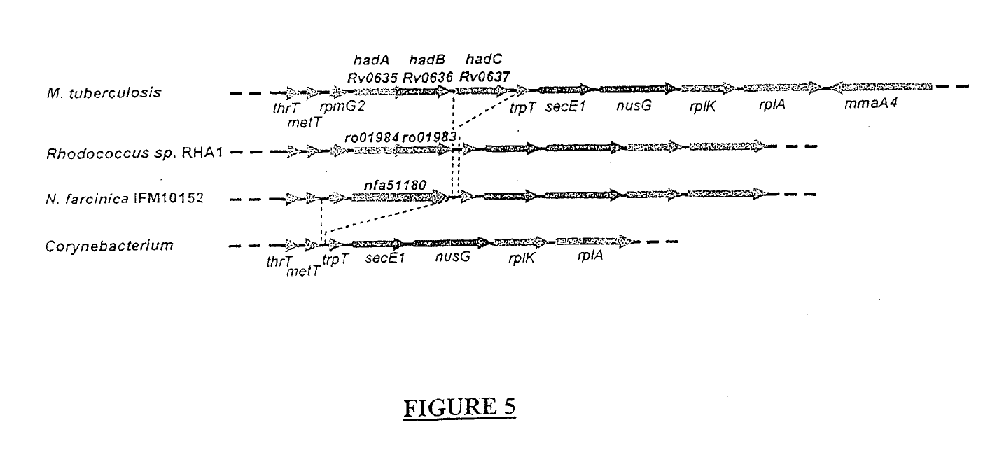

In addition, the sequence of other Corynebacterinae, namely Corynebacterium glutamicum, Rhodococcus sp. RHA1 and Nocardia farcinica has been analyzed. Although Corynebacterium produces short mycolic acids (C₃₂-C₃₆), Rhodococcus and Nocardia have mycolic acids of intermediate size (C₃₄-C₄₈ and C₄₄-C₆₀, respectively), exhibiting a meromycolic chain of medium length (C₂₂-C₃₀ and C₃₂-C₄₂), i.e. shorter than that observed in the Mycobacterium genus. As has already been observed for other enzymes involved in the FAS-II system, it has not been possible to detect polypeptides similar to Rv0635, Rv0636 and Rv0637, in Corynebacterium. This is consistent with the fact that its mycolic acid biosynthesis pathway does not include a fatty acid elongation step. Proteins orthologous to Rv0635 and Rv0636 are present in Rhodococcus and Nocardia (see results above), as observed for the InhA, MabA and KasA proteins. On the other hand, no orthologue of the Rv0637 protein is present, just as for KasB. It should be noted that the genes equivalent to Rv0635 and Rv0636 are fused into a single long gene in Nocardia (FIG. 5).

The Rv0635, Rv0636 and Rv0637 proteins, and also the proteins having the identity or similarity percentages specified above, can advantageously serve as a target for screening antibodies of use as anti-infective medicaments.

The expression “x % identity between a polypeptide P of length at least equal to that of the reference sequence, and a reference sequence” is intended to mean that, when the two sequences are aligned, x % of the amino acids of P are identical to the corresponding amino acid of said reference sequence.

The expression “x % similarity between a polypeptide P of length at least equal to that of the reference sequence, and a reference sequence” is intended to mean that, when the two polypeptides are aligned, x % of the amino acids of P are identical to the corresponding amino acid of said reference sequence or are replaced with an amino acid of the same group. When the polypeptide P is shorter in length than the reference sequence, the alignment is carried out over the total length of the polypeptide P. FIGS. 7-9 show the results obtained.

The term “amino acid of the same group” is intended to mean an amino acid having substantially identical chemical properties. In particular, this term is intended to mean amino acids having substantially the same charge and/or the same size and/or the same hydrophilicity or hydrophobicity and/or the same aromaticity.

Such groups of amino acids include, in particular:
(i) glycine, alanine,
(ii) isoleucine, leucine, valine
(iii) tryptophan, tyrosine, phenylalanine
(iv) aspartic acid, glutamic acid
(v) arginine, lysine, histidine
(vi) serine, threonine.

Other substitutions can be envisioned, in which one amino acid is replaced with another amino acid which is comparable but not natural (hydroxyproline, norleucine, ornithine, citrulline, cyclohexylalanine, dextrorotary amino acids, etc.).

The identity percentages and similarity percentages defined can be obtained using the BLAST program (or blast2seq, default parameters) (Tatustore et al., FEMS Microbiol. Lett. 1999, 174, 247-250) or PSI-BLAST program (Altshul et al., 1997) with a comparison window corresponding to the total length of the sequences SEQ ID Nos. 2, 4 and 6, when the polypeptide P compared is of length at least equal to that of the sequences SEQ ID Nos. 2, 4 and 6. When the polypeptide P to be compared is shorter in length than the sequences SEQ ID Nos. 2, 4 and 6, the comparison window corresponds to the total length of the polypeptide P.

According to one advantageous embodiment of the invention, said enzymes are selected from the group constituted of: the homomultimer of Rv0636, the heterodimer or the heteromultimer Rv0635-Rv0636 and the heterodimer or the heteromultimer Rv0636-Rv0637.

Surprisingly, the enzymes comprising Rv0636 combined as a heterodimer or as a heteromultimer with Rv0635 or Rv0637 have an enoyl-CoA hydratase activity in vitro.

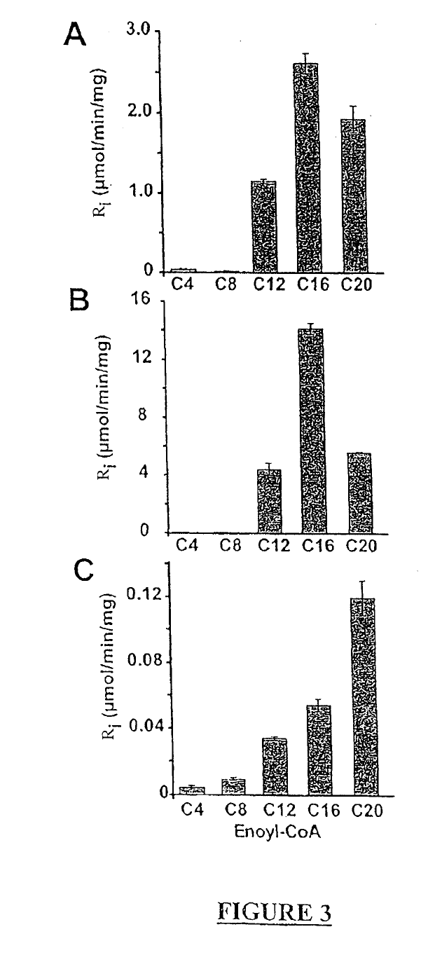

In particular, these two heterodimers effectively catalyze the hydration of 2-trans-enoyl-CoA species with chain lengths of C₁₂-C₂₀, whereas no activity is detected in the presence of short-chain substrates (C₄-C₈). They therefore exhibit a specificity for substrates of size greater than or equal to 12 carbon atoms, a property comparable with the InhA, KasA and KasB proteins of the mycobacterial FAS-II system, and with the FAS-II system itself (FIG. 3).

Of course, the specificity of an enzyme according to the invention for a given size of the acyl chain of the substrates may depend on the origin (plant, parasite, bacterium, mitochondrion) of said enzyme.

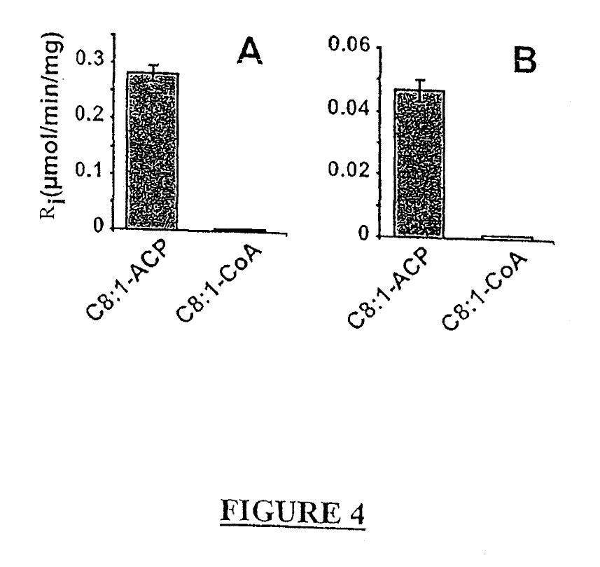

According to another advantageous embodiment of the invention, the (3R)-hydroxyacyl and/or 2-trans-enoyl substrate of said enzymes is preferably a substrate having an acyl chain of length greater than or equal to C₈, preferably C₁₂-C₂₀, derived from ACP (FIGS. 3 and 4).

Rv0636 can combine with a partner which does not comprise a hydratase 2 catalytic motif. Thus, the partner protein (Rv0635 or Rv0637) is probably present in order to stabilize the long acyl chain of the substrate.

The combining of Rv0636 with Rv0635 (so as to form a heterodimer or a heteromultimer) induces a different specificity, but also a much greater specific activity in comparison with that obtained with the Rv0636-Rv0637 heterodimer (FIG. 3). The latter enzyme exhibits an increasing activity according to the increase in chain length of the 2-trans-enoyl-CoA substrates (C₁₂-C₂₀), with apparent optimum activity for the C₂₀ substrate (FIG. 3). The specificity profiles for Rv0635-Rv0636 and Rv0636-Rv0637 are comparable to those obtained for certain proteins of the mycobacterial FAS-II system and show that they belong to the FAS-II system. The fact that the heterodimers exhibit a much higher activity in the presence of ACP derivative than of CoA derivative confirms this membership (FIG. 4). This would explain the weak specific activity of these enzymes in the presence of CoA derivatives.

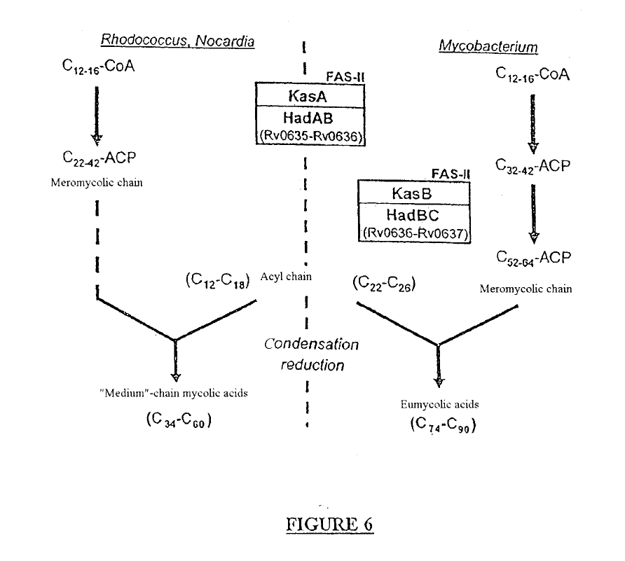

In addition, the fact that the specificity of substrates for Rv0636-Rv0637 is shifted toward the long chains shows that it could be involved, like KasB, in the final steps of elongation of the meromycolic chain, and take over from Rv0635-Rv0636 (FIG. 6). This hypothesis is reinforced by the fact, in particular, that no orthologue of Rv0637 is found in the sequenced species of Nocardia and Rhodococcus. This scheme is also found for KasB. It would explain the weak general specific activity of Rv0636-Rv0637 (compared with Rv0635-Rv0636), which then metabolize very-long-chain substrates (up to C₆₀ approximately) (FIG. 6).

Surprisingly, the results obtained show that the two heterodimers or multimers Rv0635-Rv0636 and Rv0636-Rv0637 are involved in the dehydration step of the mycobacterial FAS-II system, and more specifically that Rv0635-Rv0636 appears to be involved in the first elongation cycles, whereas Rv0636-Rv0637 appears to be involved in the later cycles.

The inventors have produced and purified the various dimers or multimers in recombinant form and have determined their catalytic activity.

The results obtained by the inventors show that these various dimers actually correspond to the (3R)-hydroxy-acyl-ACP dehydratase involved in the third, dehydration step of the mycobacterial FAS-II system and that they are key enzymes in the assembly of the envelope of mycolata. Experiments for predicting essentialness in Mycobacterium tuberculosis suggests that the Rv0635 and Rv0636 genes are essential for the viability of this species (Sassetti et al,. 2003).

Enzymes belonging to the family of hydratases 2 in the form of heteromultimers (for example, of heterodimers), composed of units with a single hotdog fold corresponding to a catalytic subunit and to a subunit without a catalytic site, have never previously been described.

In fact, in the prior art, there has only been a structure of “asymmetrical” type described for the double hotdog hydratase domain of the eukaryotic MFE-2 proteins and predicted (by modeling) for the Rv3398c hydratase/dehydratase enzyme of M. tuberculosis, but, in these two cases, the structure is composed of a single polypeptide having two structural domains, one catalytic, the other noncatalytic, which are covalently bonded. The original structure of the enzymes according to the invention may favor the discovery of specific ligands that are potentially inhibitors of these enzymes.

A subject of the present invention is also the poly-nucleotides encoding the various proteins as defined above, especially the Rv0635, Rv0636 and Rv0637 proteins; in particular, the polynucleotide of SEQ ID No. 1 encodes the Rv0636 protein; the polynucleotide of SEQ ID No. 3 encodes the Rv0635 protein and the poly-nucleotide SEQ ID No. 5 encodes the Rv0637 protein; the polynucleotide comprising the sequence which encodes an RV0636 protein and the sequence which encodes an Rv0635 protein and/or an Rv0637 protein.

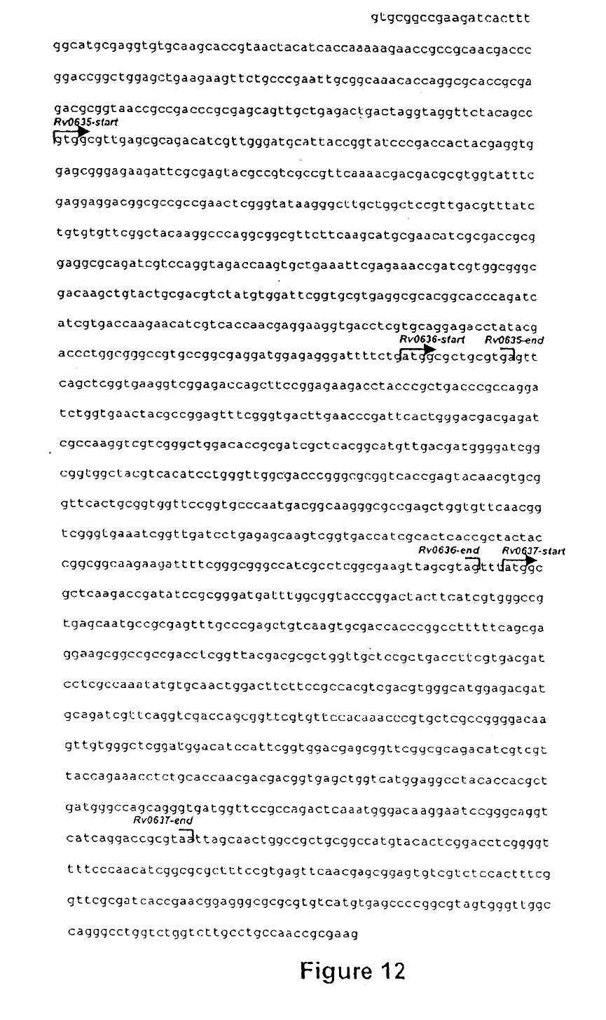

The subject of the present invention is also a poly-nucleotide represented in the sequence SEQ ID No. 7 of the sequence listing attached in the annex; it comprises the ORFs encoding the Rv0635, Rv0636 and Rv0637 proteins located between positions 73190 and 73325 of the genome of M. tuberculosis H37Rv. The chromosomal coordinates of the ORFS in said poly-nucleotide are the following: Rv0635: 731930-732406 (positions 201-677 of SEQ ID No. 7), Rv0636: 732393-732821 (positions 664-1092 of SEQ ID No. 7); Rv0637: 732825-733325 (positions 1096-1596 of SEQ ID No. 7) (FIG. 12).

A subject of the present invention is also an expression vector comprising a polynucleotide as defined above, and in particular a polynucleotide encoding an Rv0636 protein in accordance with the invention, alone or combined with a polynucleotide encoding the Rv0635 protein and/or the Rv0637 protein, and also a prokaryotic or eukaryotic host cell transformed with said expression vector.

The vector used is any vector of the prior art.

Said vector may also comprise regulatory sequences for the expression of the protein encoded by the poly-nucleotide (promoter, stop site, etc.).

The introduction of the polynucleotide or of the expression vector into the modified host cell can be carried out by any known method, such as, for example, transfection, infection, fusion, electroporation, microinjection or biolistics.

A subject of the present invention is also a method for producing an enzyme in accordance with the invention, characterized in that it comprises culturing a host cell in accordance with the invention in a suitable medium and purifying said enzyme from said culture.

Advantageously, said purification is carried out by affinity chromatography, for example IMAC, and/or exclusion chromatography. By way of nonlimiting example, mentioned may be made of the IMAC Ni-Sepharose FF column and the HiLoad 16/60 Superdex 75 prep grade column.

A subject of the present invention is also a method for inhibiting the biosynthesis of compounds of the envelope of mycolata, characterized in that it comprises inhibiting the expression or the activity of at least one of the enzymes as defined above, selected from the group constituted of the Rv0636 protein, the Rv0635 protein, the Rv0637 protein and/or the protein complexes as defined above (dimers and multimers comprising the Rv0636 protein). For example, such inhibitors may be antibodies directed against these proteins, antisense RNAs or interfering RNAs targeting the genes encoding these proteins, molecules which bind to the active site or to the substrate-binding site of these proteins, or, in particular, compounds having a phenylbenzopyrone structure, such as flavonoids (Brown et al., 2007).

According to one advantageous embodiment of said method, it comprises:
a) incubating a suitable substrate selected from the group constituted of (3R)-hydroxyacyl-ACPs, trans-2-enoyl-ACPs, (3R)-hydroxyacyl-CoAs and trans-2-enoyl-CoAs, with an enzyme in accordance with the invention, in the presence or absence of the test molecule, and
b) comparing the activity of the enzyme according to the invention in the presence and absence of the test molecule.

The subject of the present invention is also a method for screening for ligands which inhibit an enzyme according to any one of claims 1 to 7 or a protein selected from the group constituted of the Rv0635, Rv0636 and Rv0637 proteins, characterized in that it comprises a step of identifying the inhibition of the expression or of the activity of said enzyme or of said protein.

The inhibition of the hydratase/dehydratase activity may be carried out by any method known to those skilled in the art, and in particular by measuring a variation in absorbance linked to the disappearance of the substrate or to the appearance of a product, at an appropriate wavelength or by means of a tandem reaction (secondary enzyme or antibody that will detect the residual amount of substrate or the amount of product formed).

For example, the detection of the inhibitory capacity of said test molecule is carried out by:
B1) measuring the variation in absorbance linked to the disappearance of the substrate or to the appearance of the product formed, at 263 nm;
B2) determining the control enzymatic activity, in the absence of said test molecule;
B3) determining the enzymatic activity in the presence of said test molecule, and
B4) comparing the measurements obtained in B2) and in B3).

Thus, the test for inhibition of the proteins constituted of at least one of the proteins Rv0635, Rv0636 and Rv0637 or of the enzymes as defined above, by potential inhibitors, can be easily and rapidly monitored by spectrophotometry, by following the hydration of the trans double bond in the 2-position of the trans-2-enoyl substrates or the dehydration of the (3R)-hydroxyacyl substrates, at 263 nm. The inhibition constants (Ki) and the mechanism of inhibition (competitive inhibition, noncompetitive inhibition, incompetitive inhibition, irreversible inhibition, slow binding, etc.) for each molecule can be deduced therefrom. In addition, tests for ligand binding to the proteins can also be carried out by spectrofluorimetry, by virtue of the presence of Trp residues, in particular in the substrate binding site. After excitation at 303 nm, the variation in intensity of fluorescence emission at the maximum emission makes it possible to detect the binding of a ligand and to deduce therefrom the dissociation constant (Kd). The simplicity of these methods of measurement, and the relatively small volumes that they require, should enable miniaturization of the inhibition or ligand-binding tests, for the automatic high-throughput screening of combinatorial libraries, by virtue of an automated device that has a spectrophotometer or a spectrofluorimeter.

Owing to its nature predicted to be essential for viability, and to the specificity of its function, the (3R)-hydroxyacyl-ACP dehydratase of the mycobacterial FAS-II system constitutes an excellent potential target for the desired new antimycobacterial medicaments, which in particular act on the growth and/or the viability of pathogenic mycobacteria (Mycobacterium tuberculosis, M. africanum, M. leprae, M. ulcerans, M. microti, M. bovis, M. abscissus, M. avium, M. fortuitum, M. kansasii, etc.) or of new medicaments which act on the growth and/or the viability of genera related to mycobacteria belonging to the Corynebacterineae (for example, Nocardia, Rhodococcus, Gordona, etc.).

The subject of the present invention is also the use of an enzyme as defined above, for screening for antibiotics that are active on microorganisms containing a FAS-II system, in which the dehydration step is catalyzed by a dehydratase containing a hydratase 2 motif [YF]-x(1,2)-[LIVG]-[STGC]-G-D-x-N—P-[LIV]-H-x(5)-[AS] (SEQ ID No. 15) (in which x(n) represents n amino acids, said amino acids being any amino acid, and the amino acids between square brackets representing alternatives) or a catalytic motif, derived from the hydratase 2 motif and constituted at least of the basic motif D-x(4)-H, in which x(4) represents 4 amino acids, said amino acids being any amino acid, or of a motif similar to the basic motif.

According to one advantageous embodiment of this use, the hydratase 2 motif is the motif Y-A-G-V-S-G-D-L-N—P—I—H—W-D-D-E-I-A (SEQ ID No. 16).

According to another advantageous embodiment of this use, said microorganisms are Corynebacterinae, preferably bacteria of the Rhodococcus, Nocardia and Mycobacterium genera, preferably the Mycobacterium genus.

The subject of the present invention is also a method for screening for ligands of a protein (preferably RV0635, Rv0636 and Rv0637, in monomeric form, and more preferably Rv0636) or of an enzyme in accordance with the invention, characterized in that it comprises:
bringing the protein or the enzyme as defined above into contact with a reaction medium comprising the potential ligand to be tested,
after excitation at 303 nm, measuring the variation in fluorescence intensity at the maximum emission,
and on the basis of this measurement, detecting the binding of said ligand to one or more of the proteins constituting the enzyme as defined above.

The subject of the present invention is also a method for screening for ligands of a protein (preferably Rv0635, Rv0636 and Rv0637, and more preferably Rv0636) or of an enzyme in accordance with the invention, characterized in that it comprises the following steps:
bringing a protein or a enzyme according to the invention into contact with the ligand to be tested, and
analyzing the complex formed in the soluble phase between said enzyme and said ligand, in particular by NMR and/or by fluorescence.

In addition to the above arrangements, the invention also comprises other arrangements which will emerge from the description which follows, which refers to exemplary embodiments of the invention and also to the attached drawings, in which:

FIG. 1 represents the fatty acid cycle by the FAS-II system in mycobacteria. n≧8. The name of the enzymes catalyzing similar steps in E. coli is mentioned between parentheses. AcpM: mycobacterial ACP involved in the FAS-II system;

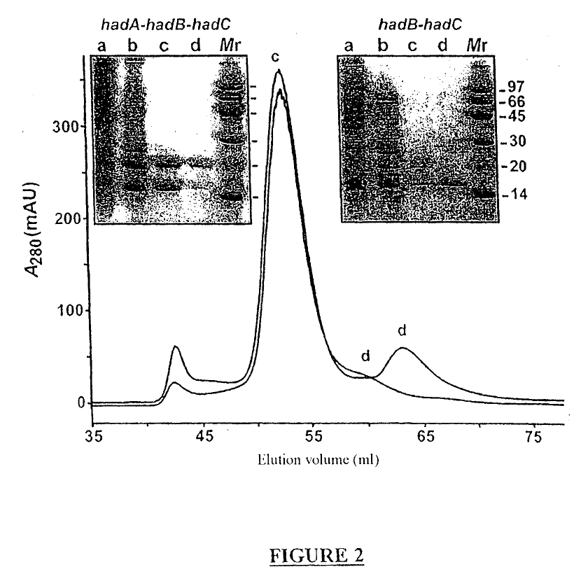

FIG. 2 illustrates the SDS-PAGE analyses of chromatography fractions on a nickel column and on an exclusion column during the purification of the products of the hadA-hadB (Rv0635-Rv0636) and hadB-hadC (Rv0636-Rv0637) genes obtained from two recombinant strains of E. coli. Lanes: a: total soluble proteins; b: pool of fractions after Ni-sepharose column; c, d: fractions after Superdex S75 column; Mr: size markers. Size of monomeric proteins: H-HadA (H-Rv0635); 18.3 kDa; HadB (Rv0636), 14.8 kDa; H-HadB (H-Rv0636), 15.9 kDa; HadC (Rv0637), 18.9 kDa;

FIG. 3 represents the specificity profiles of the heterodimeric enzymes Rv0635-Rv0636 and Rv0636-Rv0637 for the 2-trans-enoyl-CoAs of various chain lengths. The data are means of initial rates with standard deviations. The larger standard deviations observed for the long-chain substrates are due to problems of solubility. The tests were carried out in the presence of fixed concentrations of substrate and of enzyme in 100 mM sodium phosphate buffer, pH 7.0. The activities detected for the two heterodimers in the presence of short-chain (C₄, C₈) substrates are not significantly different than the activities detected in the control experiments in the absence of enzyme. The concentration of each enzyme was adjusted so as to obtain optimal conditions for measuring the initial rate. Specificity of the Rv0635-Rv0636 heterodimer in the presence of 2.5 μM (A) or 25 μM (B) of 2-trans-enoyl-CoA. Specificity of the Rv0636-Rv0637 heterodimer in the presence of 25 μM (C) of 2-trans-enoyl-CoA;

FIG. 4 represents the comparison of the specific activity of the Rv0635-Rv0636 and Rv0636-Rv0637 heterodimers in the presence of 2-trans-octenoyl-ACP and of 2-trans-octenoyl-CoA. The data are means of initial rates with standard deviations. The tests were carried out in the presence of 2 μM of substrate, and of fixed concentrations of Rv0635-Rv0636 (80 nM) (A) or RV0636-Rv0637 (543 nM) (B) enzymes, and monitored by spectrophotometry at 263 nm. The responses obtained for the Rv0635-Rv0636 and Rv0636-Rv0637 heterodimers in the presence of 2-trans-octenoyl-CoA are not significantly different than the response obtained for the control experiments (in the absence of enzyme);

FIG. 5 illustrates the chromosomal organization of all of the Rv0635, Rv0636 and Rv0637 genes or of the orthologous genes in various Corynebacterinae. The products of the genes: thrT, t-RNA-Thr; metT, t-RNA-Met; rmpG2, probable 50S ribosomal protein L33; trpT, t-RNA-Trp; secE1, probable translocase preprotein; nusG, probable anti-termination of transcription protein; rp1K, rp1A, probable 50S ribosomal proteins L11 and L1; mmaA4, mycolic acid (MA) methyltransferase. ro01984 plus ro01983 and the fusion gene nfa51180 are orthologues of hadA and hadB in Rhodococcus sp. and Nocardia farcinica, respectively. The schemes are based on the sequenced genomes of Rhodococcus, Nocardia and Corynebacterium and of the strains of the Mtb complex and of M. leprae. It should be noted that variations downstream of the rp1A gene exist in other genomes of mycobacteria which have been sequenced;

FIG. 6 illustrates the model of the roles of the Rv0635-Rv0636 (HadAB) and Rv0636-Rv0637 (HadBC) heterodimers in the mycolic acid biosynthesis pathway. This model proposes that the Rv0635-Rv0636 (HadAB) enzyme is involved, just like the KasA enzyme, in the early steps of fatty acid elongation catalyzed by the FAS-II system, resulting in the formation of meromycolic chains of intermediate size (C₂₂-C₄₂) and, consequently, in the formation of medium-chain-length mycolic acids found in Rhodococcus and Nocardia. In the Mycobacterium genus, the Rv0636-Rv0637 (HadBC) enzyme is thought to subsequently be involved, just like the KasB enzyme, in the late steps of elongation of the meromycolic chains of intermediate size, so as to give molecules of whole size (C₅₂-C₆₄), catalyzed by the FAS-II system, leading to the synthesis of eumycolic acids (C₇₄-C₉₀). Only some of the proteins of the FAS-II system are mentioned in this figure;

FIGS. 7, 8 and 9 represent the percentages of identity and the percentages of similarity of the Rv0635, Rv0636 and Rv0637 proteins with the orthologous proteins encountered in mycobacteria, in Rhodococcus and Nocardia, the genome of which has been sequenced (updated: Oct. 31, 2006). When the genome has not yet been annotated, the name of the contig containing the gene in question, and also the position of the gene on the contig (in base pairs), are mentioned. The total number of amino acids (aa) on which the sequence alignment was carried out (BLASTP software—protein/protein) is mentioned, as is the score obtained, “E value”. These FIGS. 7, 8 and 9) illustrate the ubiquity of the Rv0635, Rv0636 and Rv0637 proteins in mycobacteria;

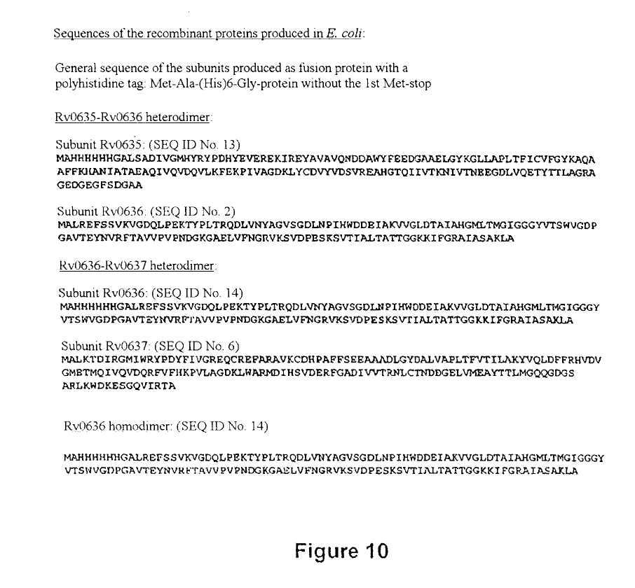

FIG. 10 illustrates the polypeptide sequences corresponding to the nucleotide sequences cloned in the recombinant E. coli strains;

FIG. 11 illustrates the alignment of the amino acid sequences of the Rv0635 (accession number: P96926), Rv0636 (accession number: P96927) and Rv0637 (accession number: P96928) proteins, carried out using the Clustal W program. Rv0636 exhibits 13% sequence identity with Rv0635 and 15% with Rv0637. Rv0636 and Rv0637 exhibit 45% identity with one another. The black shading and gray shading indicate, respectively, the residues strictly conserved and similar. The hydratase motif ([YF]-x(1,2)-[LIVG]-[STGC]-G-D-x-N—P-[LIV]-H-x(5)-[AS]) of Rv0636 is indicated by stars;

FIG. 12 represents the coding sequences of the Rv0635-Rv0636-Rv0637 operon in M. tuberculosis. The start and the end of each ORF is indicated by an arrow or a line, respectively. The Rv0635 and Rv0636 genes overlap;

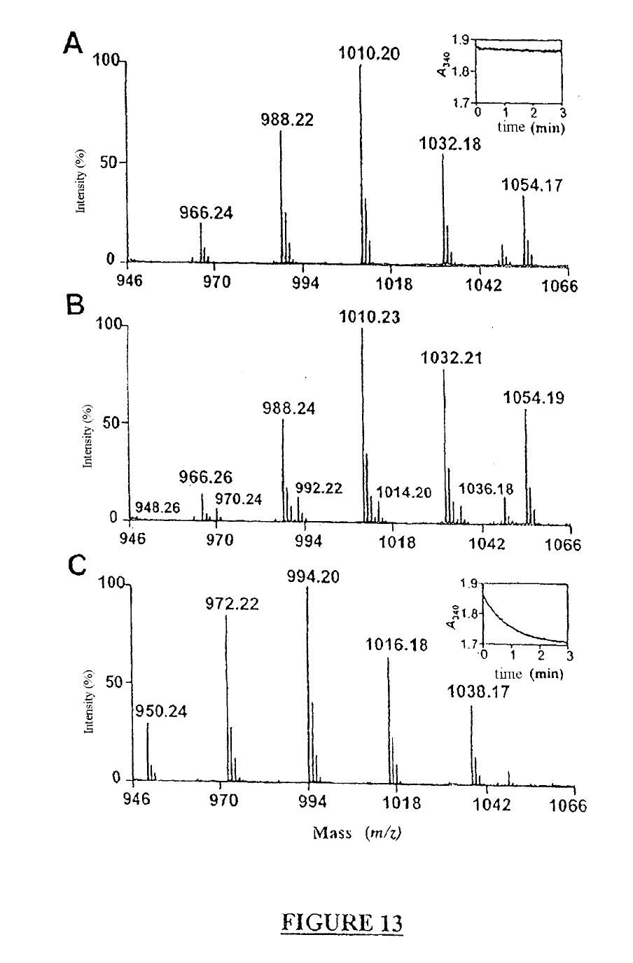

FIG. 13 represents the tests for coupling of the Rv0635-Rv0636 (HadAB) heterodimer in the presence of MabA and InhA. It illustrates the MALDI-TOF MS analyses of the reaction medium containing 3-ketododecanoyl-CoA, NADPH, NADH, MabA plus InhA (A) or plus 280 nM of the Rv0635-Rv0636 heterodimer (B) or plus the Rv0635-Rv0636 heterodimer and InhA (C). For (A) and (B), the peaks at 966, 988, 1010, 1032 and 1054 m/z, respectively, represent the [M+H]⁺, [M+Na]⁺, [M−H+2Na]⁺, [M−2H+3Na]⁺ and [M−3H+4Na]⁺ ions of 3-hydroxydodecanoyl-CoA (product of MabA). For (B), the minor peaks at 948, 970, 992, 1014 and 1036 m/z represent the [M+H]⁺ ion and the mono- to tetrasodium addition products of the unsaturated species, dodecanoyl-CoA. For (C), the peaks at 950, 972, 994, 1016 and 1038 represent the [M+H]⁺ ion and the mono- to tetrasodium addition products of the saturated species, dodecanoyl-CoA. The graphs added in (A) and (C) illustrate the kinetics of the respective reactions at 340 nm (oxidation of the InhA coenzyme, NADH). The three spectra correspond to the 3 min reaction time;

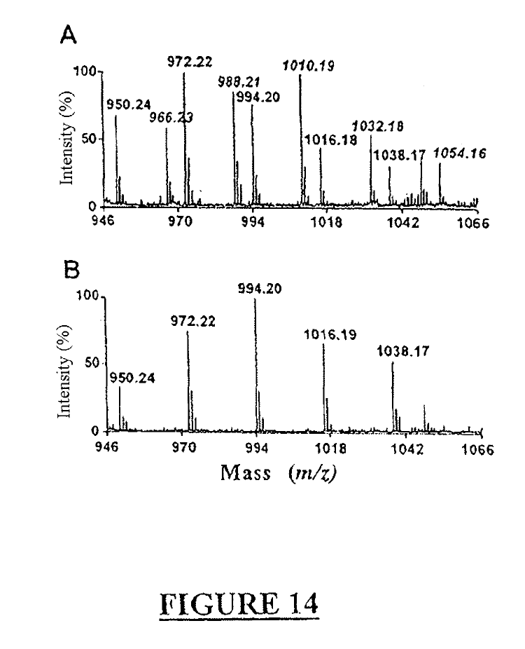

FIG. 14 represents the tests for coupling of the Rv0636-Rv0637 (HadBC) heterodimer in the presence of MabA and InhA. They illustrate the MALDI-TOF MS analyses of the reaction medium containing 3-ketodo-decanoyl-CoA, NADPH, NADH, MabA, plus InhA and the Rv0636-Rv0637 (HadBC) heterodimer (1.65 μM), after 3 h (A) and 24 h (B) of reaction. For the meaning of the peaks, see the legend of FIG. 13 above. The control experiments (without heterodimer or without InhA) are similar to those obtained in the presence of the Rv0635-Rv0636 (HadAB) heterodimer, although the ion peaks have different intensities.

### EXAMPLE 1

### Bioinformatic Analyses and Molecular Modeling

1.A. Sequence Analyses

The genome sequence analyses were carried out using the Internet servers of the Sanger Institute and TubercuList for Mycobacterium tuberculosis H37Rv (Cole et al., 1998) and the “National Center for Biotechnology Information” (NCBI) Internet site for the other genomes (www.ncbi.nlm.nih.gov/genomes/lproks.cgi). The sequence alignments were carried out using the BLAST or PSI-BLAST, Clustal W version 1.8 or MultAlin software with the default parameters (Altschul et al., 1997; Corpet, 1998; Thompson et al., 1994).

1.B. Definition of the FabA/FabZ Specific Common Motif

The PRATT version 2.1 program of the PROSITE database (Jonassen et al., 1995) was used with the default parameters to define the two distinct specific motifs of the FabZ or FabA annotated proteins, based on two independent lists of 36 protein sequences of FabZ type, and 24 sequences of FabA type [including the FabZ1 protein of Enterococcus faecalis, the sequence of which is more related to the FabZ proteins but which has an FabA activity (dehydratase/isomerase) (Wang & Cronan, 2004)]. Each motif was then used as a probe against the databanks proposed (Swiss-Prot, EMBL and TrEMBL) by the ScanProsite software (default parameters) in order to evaluate the specificity of the motif for the two protein families (Gattiker et al., 2002). A common consensus motif specific for FabA/FabZ was then determined manually on the basis of the two previously defined motifs. This common consensus motif was used as a probe against all the protein databanks or against the banks of proteins predicted for various species of the Mycobacterium genus that are available, by this software. A control experiment was carried out using the same strategy as that described above, but with the proteins of the FabG family. This analysis against the predicted proteins of the Mycobacterium genus made it possible to pick out the annotated proteins such as FabG of the mycobacterial species and it showed that mycobacterial proteins could be detected by this strategy.

### EXAMPLE 2

### PCR Amplification and Cloning of the Genes Encoding Rv0635, Rv0636 and Rv0637

2.1 PCR Amplification

Rv0636

The gene encoding the Rv0636 protein was amplified by PCR (Polymerase Chain Reaction) in two steps, using the total DNA of the M. tuberculosis strain H37Rv.

In a first PCR reaction, the pair of primers 5′-GAT TTT CTG ATG GCG CTG CGT GAG TTC-3′ (sense primer) (SEQ ID No. 8) and 5′-CGG TCT TGA GCG CCA TAA ACT A-3′ (anti-sense primer) (SEQ ID No. 9) was used. In a second PCR reaction, an N-terminal tag of 6 histidines was introduced using the sense primer 5′-ATG GCT CAT CAT CAT CAT CAT CAT GGT GCG CTG CGT GAG TTC AGC TCG G-3′ (SEQ ID No. 10). The PCR reactions were carried out with the PfuUltra polymerase (Stratagene).

Rv0635-Rv0636

In order to coexpress the Rv0635 and Rv0636 proteins, the nucleotides 731 930 to 733 325 of the genome of the M. tuberculosis strain H37Rv, containing the three genes Rv0635, Rv0636 and Rv0637, were amplified by PCR using the cosmid MTCY20H10 (http://genolist.pasteur.fr/Tuberculist). An N-terminal tag of 6 histidines were simultaneously introduced in the PCR reaction by using the pair of primers 5′-ATG GCT CAT CAT CAT CAT CAT CAT GGT GCG TTG AGC GCA GAC ATC G-3′ (sense primer) (SEQ ID No. 11) and 5′-CAG TTG CTA ATT ACG CGG TC-3′ (antisense primer) (SEQ ID No. 12), and the PfuUltra polymerase (Stratagene).

Rv0636-Rv0637

In order to coexpress the Rv0636 and Rv0637 proteins, a DNA fragment containing the two genes Rv0636 and Rv0637 was amplified using the cosmid MTCY20H10 (http://genolist.pasteur.fr/Tuberculist). An N-terminal tag of 6 histidines were simultaneously introduced in the PCR reaction by using the pair of primers 5′-ATG GCT CAT CAT CAT CAT CAT CAT GGT GCG CTG CGT GAG TTC AGC TCG G-3′ (sense primer) (SEQ ID No. 13) and 5′-CAG TTG CTA ATT ACG CGG TC-3′ (antisense primer) (SEQ ID No. 14), and the PfuUltra polymerase (Stratagene).

2.2 DNA Ligation

After the addition of a 3′ protruding poly(A) end by incubation with Taq polymerase (New England Biolabs), the genes amplified with the pCR T7 TOPO or pEXP5-CT/TOPO vector (Invitrogen) were ligated under the conditions specified by the supplier.

2.3 Cloning

The cloning was carried out in E. coli TOP10 (Invitrogen), and the exactness of the genes isolated was controlled by DNA sequence analysis.

### EXAMPLE 3

### Expression and Purification of the Rv0635, Rv0636 and Rv0637 Proteins

The expression of the genes was carried out with E. coli BL21-AI (Invitrogen) transformed with Rv0636, and with E. coli BL21 Star (DE3) (Invitrogen) transformed with Rv0636-Rv0637 or with Rv0635-Rv0636-Rv0637, the genes being integrated into the constructs described below:
E. coli

BL21 Star (DE3)/pEXP5-CT/TOPO::H-Rv0635-Rv0636-Rv0637;
E. coli BL21 Star (DE3)/pEXP5-CT/TOPO::H-Rv0636-Rv0637 and
E. coli BL21-AI/pCR T7 TOPO::H-Rv0636.

The bacteria were cultured in LB medium (Luria Broth Base, DIFCO-BRL) supplemented with 50 μg/ml of ampicillin, at 37° C.

Rv0636

At an OD₆₀₀=0.7-0.9, the expression of the Rv0636 target gene was induced with 0.02% of arabinose for 3-4 hours. After centrifugation of the bacterial cultures, the cell pellet was resuspended in a lysis buffer (50 mM NaH₂PO₄, 300 mM NaCl, 10 mM imidazole, pH 8.0), and lyzed using freeze-thawing cycles. The major part of the Rv0636 protein is in insoluble form.

After centrifugation at 19 000 rpm (43 000×g) for 20 minutes, the cell-free extract was loaded onto a preequilibrated Ni Sepharose FF column (1 ml, GE Healthcare). After washing with 20 column volumes of buffer (20 mM imidazole, 50 mM NaH₂PO₄, 300 mM NaCl, pH 8.0), the protein was eluted with 5 column volumes of 250 mM imidazole in the same buffer.

The fractions containing the protein are combined, and their buffer is changed by chromatography on a PD-10 column (GE Healthcare). The enzyme is further purified on a HiLoad 16/60 Superdex 75 prep grade column (GE Healthcare) equilibrated with 50 mM HEPES buffer containing 150 mM NaCl, pH 7.5. The solution of purified protein is analyzed by Coomassie blue staining on SDS-PAGE (FIG. 2). This protein is 95% pure. It is, however, relatively unstable in solution. Glycerol is added to a final concentration of 50% (v/v) and the protein is stored at −20° C.

Rv0635-Rv0636 and Rv0636-Rv0637

At an OD₆₀₀=0.5-0.7, the expression of the Rv0635-RV0636 and Rv0636-Rv0637 genes was induced with 0.2 mM of IPTG for 3-4 hours. After centrifugation of the bacterial cultures, the cell pellet was resuspended in a lysis buffer (50 mM NaH₂PO₄, 300 mM NaCl, 10 mM imidazole, 10% (v/v) glycerol, pH 8.0) with 1 mg/ml lysozyme, 0.01 mg/ml RNAse A, 0.02 mg/ml DNAse I and 1 mM PMSF, and lyzed using the One Shot Cell disruptor (Constant Systems Ltd.).

The soluble fraction was loaded onto a preequilibrated Ni Sepharose FF column (1 ml, GE Healthcare). After washing with 20 column volumes of buffer (20 mM imidazole, 50 mM NaH₂PO₄ or HEPES, 300 mM NaCl, 10% (v/v) glycerol, pH 8.0), the protein was eluted with 5 column volumes of the same buffer comprising 250 mM imidazole.

The fractions containing the enzyme are combined, and their buffer is changed by chromatography on a PD-10 column (GE Healthcare). The enzyme is further purified on a HiLoad 16/60 Superdex 75 pre grade column (GE Healthcare) equilibrated with 50 mM HEPES, 150 mM NaCl, pH 7.5. Glycerol is added to the fractions (up to 50% v/v) and the protein solution is stored at −20° C.

The solution of purified protein is evaluated by Coomassie blue staining on SDS-PAGE (FIG. 2), and then by mass spectrometry.

### EXAMPLE 4

### Analysis of the Structure of the Rv0635, Rv0636 and Rv0637 Proteins

Rv0636 Heterodimers

During the chromatography steps described above, coelution of the Rv0635 (fused with the N-terminal polyhistidine tag) and Rv0636 proteins, and elution of the Rv0636 (fused with the N-terminal polyhistidine tag) and Rv0637 proteins was demonstrated (FIG. 2). These coelutions result from interaction, on one hand, of Rv0635 with Rv0636 and, on the other hand, of Rv0636 with Rv0637. The exclusion chromatography step, carried out at 4° C., suggested that these Rv0635-Rv0636 and Rv0636-Rv0637 heterocomplexes were tetrameric (FIG. 2). The quaternary structure of Rv0635-Rv0636 was further examined by Dynamic Light Scattering.

The Dynamic Light Scattering measurements were carried out with a DynaPro-801 TC (Protein Solutions, Inc), at a protein concentration of 20 mg/ml in 50 mM NaHEPES, 150 mM NaCl, pH 7.5, at temperatures of 8 to 20° C. The data were analyzed with Dynamics. The sample showed a monomodal size distribution.

These measurements confirm that this enzyme (33.1 kDa) forms a tetramer at low temperature (8° C.). However, at higher temperature (20° C.) it behaves like a heterodimer. The results are shown in table 1 below.

TABLE 1
Dynamic light scattering measurements for the
Rv0635-Rv0636 complex at two different temperatures.

| | Hydrodynamic | | Estimated |
|---|---|---|---|
| Temperature | radius | Polydispersion | molecular |
| (° C.) | (nm) | (nm) | weight |
| 8 | 3.4 ± 0.1 | 0.7 | 57 |
| 20 | 2.9 ± 0.1 | 0.7 | 37 |

### EXAMPLE 5

### Demonstration of the Catalytic Activity of the Enzymes In Vitro

Synthesis of Fatty Acid Derivatives

The 2-trans-enoyl-CoA was synthesized and purified according to the protocol described in Quemard at al., 1995. The synthesis of 3-cis-decenoyl-CoA was carried out as follows.

The 3-cis-decenoyl-CoA was prepared and purified from 3-cis-decenoic acid and CoA, according to the same mixed anhydride method as for 2-trans-enoyl-CoA (Goldman & Vagelos, 1961). The 3-cis-decenoyl-CoA was purified by reverse-phase HPLC using a Nucleosil C₁₈ 8×250 nm column (Bischoff Chromatography). The column was preequilibrated with a methanol/water mixture containing 20 mM of NaH₂PO₄, and the elution was carried out with a gradient of 10 to 60% of methanol in water at 1.5 ml/min. The detection was carried out by measuring the absorbance at 260 nm.

The 3-cis-decanoic acid was synthesized by oxidation of 3-cis-decen-1-ol with pyridinium dichromate in dichloromethane and dry dimethylformamide, according to the protocol described by Besra (Besra et al., 1993). It was purified by silica gel chromatography using an 80/20 (v/v) mixture of CHCl₃/CH₃OH as eluent, and characterized by ¹H-NMR spectroscopy. The 3-cis-decen-1-ol was obtained by catalytic semihydrogenation (1 atm) of the solution of 3-decyn-1-ol (158 mg) in dry diethyl ether on the Lindlar catalyst (40 mg) with vigorous stirring until the hydrogen gas (1.6 mmol) was absorbed. It was then purified by adsorption chromatography on silica gel with silver nitrate, using increasing concentrations of methanol in dichloromethane as eluent.

The 2-trans-octenoyl-ACP was synthesized from E. coli holo-ACP (Sigma®) and 2-trans-octenoic acid as previously described (Cronan & Klages, 1981).

Calibration of the Reagent Solutions

The concentrations of the solutions or substrates 2-trans-enoyl-CoA, 3-cis-decenoyl-CoA, and 2-trans-octenoyl-ACP were determined by spectrophotometry at 260 nm with the following molar extinction coefficients: for the 2-trans-enoyl-CoAs, ε₂₆₀=22 600 M⁻¹ cm⁻¹, for the 3-cis-decenoyl-CoA, ε₂₆₀=16 800 M⁻¹ cm⁻¹, or at 280 nm for the 2-trans-octenoyl-ACP (ε₂₈₀=1800 M⁻¹ cm⁻¹). a more precise calibration of the concentration of 2-trans-octenoyl-ACP was then carried out in the presence of the purified M. tuberculosis InhA enzyme (25 nM) and of NADH (at saturating concentration: 100 μM). The reaction was monitored by spectrophotometry at 340 nm, which is the wavelength at which the oxidation of NADH is monitored, and the total concentration of converted substrate was deduced.

Principle of the Enzymatic Test

The (R)-specific hydratases/dehydratases are more active in vitro in the direction of hydration than in the direction of dehydration when they are isolated from their complex. Thus, their in vitro activity is often studied in the presence of enoyl derivatives rather than (3R)-hydroxyacyl derivatives.

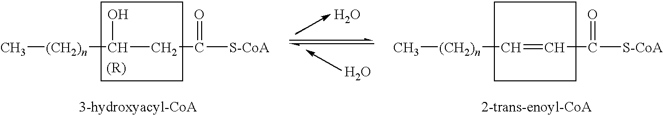

The reaction monitored is the conversion of a 2-trans-enoyl-CoA (or ACP) into (3R)-hydroxyacyl-CoA (or ACP) corresponding to the hydration reaction, or the conversion of a (3R)-hydroxyacyl-CoA into 2-trans-enoyl-CoA corresponding to the dehydration reaction.

Standard Conditions

These reactions are monitored by spectrophotometry at 263 nm by means of a thermostated Uvikon 923 spectrophotometer (Kontron Instruments). They are carried out in a quartz cuvette (optical path of 1 cm) in a total volume of 700 μl at 25° C. The reaction mixture contains 100 mM sodium phosphate buffer, pH 7.0, and varying concentrations of substrates. After equilibration of the base line on the reaction mixture in the absence of the enzyme, the reactions are triggered by the addition of enzyme, and then monitored for 1.5 to 5 min. The reaction rate corresponds to the initial rate measured by plotting the tangent of the curve OD=f(time) at time zero.

The conversions corresponding to the hydration reaction and to the dehydration reaction are associated, respectively, with a decrease and with an increase in the absorbance at 263 nm, linked to the double bond conjugated to the carbonyl. A variation in OD₂₆₃ of 0.67 corresponds to a variation in concentration of 100 μM.

Determination of the Specific Hydratase/Dehydratase Activity of the Rv0635-Rv0636 and Rv0636-Rv0637 Enzymes

In order to carry out the various enzymatic tests, several concentrations of enzyme were tested, thereby making it possible to define, for each enzyme, the concentration most suitable for the measurements.

In order to determine whether the enzymes are active, the first hydration tests were carried out in the presence of 25 μM of crotonoyl-CoA or of 2-trans-octenoyl-CoA and of the following ranges of enzyme concentrations: 0.8 to 280 nM of the Rv0635-Rv0636 heterodimer, 0.8 to 543 nM of the Rv0636-Rv0637 heterodimer. As for the dehydratase activity, it was studied in the presence of 25 or 75 μM of hydroxybutyryl-CoA substrate and the same enzyme concentration as for the hydratase activity tests, i.e. 0.8 to 280 nM of the Rv0635-Rv0636 heterodimer, 0.8 to 543 nM of the Rv0636-Rv0637 heterodimer.

Determination of the Substrate Specificity of the Enzymes

The substrate specificity was studied in the presence of 2-trans-enoyl-CoA of various chain lengths (C₄-C₂₀). The initial rates were measured and compared at a very low substrate concentration in order to reduce as much as possible the solubility problems encountered with these amphiphilic compounds. The reactions were carried out in the presence of 2.5 or 25 μM of substrate; the respective concentrations of the Rv0635-Rv0636 and Rv0636-Rv0637 enzymes were the following: 80 nM and 543 nM.

Activity on C₄-C₈ Substrates

The activity of the purified Rv0635-Rv0636 and Rv0636-Rv0637 heterodimers was tested in the presence of a 2-trans-enoyl-CoA since the (R)-specific dehydratases/hydratases preferentially function in the direction of hydration, in vitro, when they are isolated from their complex.

The tests were carried out in 100 mM sodium phosphate buffer, pH 7.0, in the presence of 3-hydroxybutyryl-CoA (25 or 75 μM) or of 2-trans-octenoyl-CoA (25 μM) or of crotonoyl-CoA (25 μM), and of enzyme (Rv0635-Rv0636: 80 nM; Rv0636-Rv0637: 543 nM). Several concentrations of enzyme were tested. When no activity was detected, the maximum concentration tested is indicated. After addition of the enzyme, the reactions were monitored by spectrophotometry at 263 nM for 1.5 min.

The results show these enzymes are not active under these conditions on C₄ or C₈ substrates, since no decrease in absorbance was observed.

Activity on C₁₂-C₂₀ Substrates

The activity of the various enzymes was tested on long-chain derivatives, C₁₂-C₂₀ trans-2-enoyl-CoAs. The tests were carried out in the presence of a low substrate concentration (2.5 μM) for the Rv0635-Rv0636 heterodimer in order to reduce as much as possible the solubility problems associated with these amphiphilic molecules. For the Rv0636-Rv0637 heterodimer, the experiments were carried out in the presence of a higher substrate concentration (25 μM) since, at 2.5 μM, no activity could be detected. In order to be able to make a comparison, the study of the specificity of Rv0635-Rv0636 was also carried out at 25 μM (FIG. 3). The Rv0635-Rv0636 heterodimer exhibits a specificity profile clearly shifted toward the long chains (C₁₂-C₁₆) and it exhibits an apparent specificity centered on 2-trans-hexadecenoyl-CoA (C₁₆) (FIG. 3A). In fact, no activity was detected in the presence of short-chain (C₄-C₈) substrates. At 25 M of 2-trans-enoyl-CoA, the Rv0635-Rv0636 heterodimer exhibits a specificity profile comparable to that obtained in the presence of 2.5 μM of substrate, other than the smaller difference between the activities in the presence of the C₁₂ and C₂₀ substrates (FIG. 3D). This decrease in difference could be due to a problem of solubility inducing the formation of micelles of the long-chain C₂₀ substrate at 25 μM. For the Rv0636-Rv0637 heterodimer, similar specificity profiles were observed (FIGS. 3B, 3C). These enzymes, which are inactive under these conditions in the presence of short-chain (C₄-C₈) substrates, show a specificity for medium-chain and long-chain (C₁₂-C₂₀) substrates. But, unlike Rv0635-Rv0636, their activity gradually increases with the chain length, up to C₂₀.

These results demonstrate important differences in the level of substrate specificity of the enzymes in accordance with the invention. The apparent preference of the Rv0635-Rv0636 heterodimer for hexadecenoyl-CoA (C₁₆) is reminiscent of that of the InhA protein of the mycobacterial FAS-II system (Quemard et al., 1995) (FIG. 3F). It is interesting to note that the association of Rv0636 with Rv0635 to give a heterodimer is accompanied by a very large increase in the specific activity (roughly by a factor of 100) compared with the association of Rv0636 with Rv0637.

These results confirm that the Rv0635, Rv0636 and Rv0637 proteins are involved in the mycobacterial FAS-II elongation system since the enzymes that they form exhibit a medium-chain and long-chain (C₁₂-C₂₀) substrate specificity, like the FAS-II system and some of its already characterized proteins.

Determination of the Isomerase Activity of the Enzymes

As in certain FAS-II systems described, the dehydration step can be catalyzed either by a (3R)-hydroxyacyl-ACP dehydratase, or by a (3R)-hydroxyacyl-ACP dehydratase/isomerase of FabA type which, in vivo, converts the 2-trans-enoyls, formed by the dehydration step, into 3-cis-enoyls. The isomerase activity can be studied in vitro by studying the reverse reaction, i.e. the conversion of a 3-cis-enoyl compound into a 2-trans-enoyl compound.

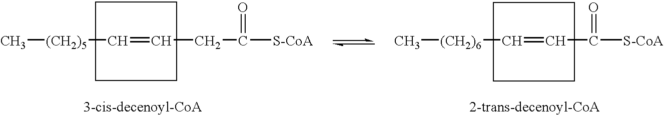

The ability of the proteins to catalyze the isomerization reaction was studied in the presence of 3-cis-decenoyl-CoA (10 μM) and of Rv0635-Rv0636 enzyme (80 nM) or Rv0636-Rv0637 enzyme (543 nM). The control experiments were carried out in the presence of 2-trans-decenoyl-CoA (10 μM) and of the same concentrations of enzyme. The reactions were monitored at 263 nm, where the increase in absorbance results from the conversion of the 3-cis-decenoyl-CoA into 2-trans-decenoyl-CoA.

No variation in absorbance was observed in the presence of 3-cis-decenoyl-CoA, whereas for the control experiments, the Rv0635-Rv0636 and Rv0636-Rv0637 enzymes exhibited a respective specific activity of 0.20 μmol/min/mg of protein and 0.02 μmol/min/mg of protein. Under the conditions tested, the enzymes studied do not exhibit any isomerase activity.

Study of the ACP-Dependency

The particularity of the FAS-II systems is that they have an ACP-dependent activity. FAS-II is the only fatty acid biosynthesis system which is ACP-dependent in mycobacteria.

The activities of the enzymes Rv0635-Rv0636 (80 nM), Rv0636-Rv0637 (311 nM) or Rv0636 alone (311 nM) were compared in the presence of 2-trans-octenoyl-ACP (2 μM) or of 2-trans-octenoyl-CoA (2 μM).

The Rv0635-Rv0636 heterodimer exhibited a specific activity in the presence of 2-trans-ocetenoyl-ACP of 0.28 μmol/min/mg, whereas no significant activity was detected in the presence of the CoA derivative under these conditions (FIG. 4A). As for the Rv0636-Rv0637 heterodimer, the specific activity detected in the presence of 2-trans-octenoyl-ACP (2 μM) was 0.047 μmol/min/mg of protein, whereas in the presence of 2-trans-octenoyl-CoA (2 μM), its activity was not significant compared with the response of the control experiment (no enzyme) (FIG. 4B). Interestingly, no activity of the Rv0636 homodimer could be detected in the presence of octenoyl-ACP.

Tandem Reaction with MabA and InhA

The inventors tested the ability of the two heterodimers to function in a coupled reaction in the presence of MabA and InhA, which are the two reductases of FAS-II that catalyze the reactions upstream and downstream of the dehydration step in the cycle (FIG. 1). Firstly, the reaction of MabA (73 nM) was carried out in the presence of NADPH (64 μM) and of 3-ketodecanoyl-CoA (C₁₂, 53 μM), and, after the reaction (monitored at 340 nm) was complete, one of the heterodimers (either 280 nM of Rv0635-Rv0636 or 1.65 μM of Rv0636-Rv0637) plus InhA (1.4 μM) were added in the presence of NADH (259 μM). Control experiments in the absence of the heterodimer or of InhA were carried out.

The reaction media were analyzed by MALDI-TOF mass spectrometry (MALDI-TOF MS). The reaction media were first of all diluted 10 times in water. The samples (1 μl) were deposited onto the target plates, mixed with 1 μl of matrix [10 mg/ml of 2,5-dihydroxybenzoic acid in water:acetonitrile, 8:2 (v/v)] and left to crystallize at ambient temperature. The analyses of the coupling reactions were carried out in reflectron mode on a 4700 Analyser mass spectrometer (Applied Biosystems) equipped with an Nd:YAG laser (wavelength of 355 nm, pulse duration<500 ps and 200 Hz repetition rate). 2500 shots were accumulated in positive or negative ion mode and the mass spectrometry data were acquired using the instrument default calibration.

The MALDI-TOF mass spectrometry analysis of the reaction media demonstrates that dodecanoyl-CoA, the saturated product of InhA, is formed very rapidly (in 3 min) in the presence of Rv0635-Rv0636 (FIG. 13C). This biosynthesis did not take place in the absence of the heterodimer (FIG. 13A). In the absence of InhA, a small proportion of dodecenoyl-CoA intermediate, a product of the hydration reaction catalyzed by Rv0635-Rv0636, appeared. This type of profile is reminiscent of what has been described for other hydroxyacyl-ACP dehydratases: in the absence of enoyl reductase, the reaction equilibrium is in favor of the hydration reaction. By comparison, the compete reaction in the presence of the Rv0636-Rv0637 heterodimer occurred very slowly: the product of InhA began to appear only after min, and the reaction was complete between 3 and 24 h of incubation (FIG. 14). This weak activity is correlated with the relatively low specific activity of Rv0636-Rv0637 in the presence of medium-chain CoA derivatives (see above).

These results as a whole strongly suggest that the Rv0635, Rv0636 and Rv0637 proteins are involved in the FAS-II elongation system. The two heterodimers, Rv0635-Rv0636 and Rv0636-Rv0637, are active only in the presence of ACP derivatives at the low substrate concentration used. These data are reminiscent of what has been observed for the enzymes of the FAS-II systems that have been described, in particular for the mycobacterial enzymes InhA, KasA and KasB, and also for the (3R)-hydroxyacyl-ACP dehydratases of other organisms which, although they show a preference for ACP derivatives, remain active in the presence of CoA derivatives. Furthermore, the two heterodimers exhibit a (3R)-hydroxyacyl dehydratase activity in the presence of the two reductases of the FAS-II complex.

### Summary of the Properties of the Rv0636 Protein

The Rv0636 protein has the following properties:

(i) the ubiquity of the protein among the mycobacterial species and also the presence in related genera, insofar as mycolic acids are compounds characteristic of the Corynebacterineae; (ii) the absence of the protein in Corynebacterium; (iii) the presence in the region of ORF on the chromosome of genes described as being involved in mycolic acid metabolism, and (iv) the presence of a catalytic motif.

More specifically, the sequence encoding the Rv636 protein is located close to the group of genes mmaA1-4 involved in the biosynthesis of oxygenated mycolic acids in M. tuberculosis. In addition, this protein has a conserved characteristic catalytic sequence, known as hydratase 2 motif (FIG. 12), which includes the motif Y-A-G-V—S-G-D-L-N—P—I—H—W-D-D-E-I-A (SEQ ID No. 16). Thus, this protein belongs to the hydratase 2 sub-family. The hydratase 2 motif was first of all described in enoyl-CoA hydratases and has also been found in the (3R)-hydroxyacyl dehydratase domain of the FAS-I systems. This hydratase 2 motif has recently been observed in the (3R)-hydroxyacyl-ACP dehydratase of the FAS-II system in yeast mitochondria. Finally, the Rv0635 gene overlaps the Rv0636 gene and the Rv0637 gene is shifted by 4 by relative to the Rv0636 gene. These three genes probably form an operon; the proteins encoded by this set of genes are predicted to have an SHD structure, i.e. a “single hotdog” fold.

Summary of the Properties of the Rv0636 and Rv0637 Proteins

The Rv0635 and Rv0637 proteins have the following properties:

(i) they are ubiquitous among the mycobacteria and only Rv0635 is found in related genera (Corynebacterinae); (ii) they are absent from Corynebacterium; (iii) their predicted structure corresponds to an SHD fold; (iv) but they do not comprise a catalytic motif; (v) they have the property of associating, independently with Rv0636, forming heterodimers or multimers.

Summary Concerning Enzymes Comprising the Rv0635, Rv0636 and Rv0637 Proteins

Surprisingly, the enzymes comprising the Rv0635, Rv0636 and Rv0637 proteins have the following properties:

(i) they form quaternary structures corresponding to dimers or multimers of Rv0636 alone, of the association Rv0635-Rv0636 and of the association Rv0636-Rv0637;

(ii) the quaternary structures comprising a catalytic subunit and a noncatalytic subunit (Rv0635-Rv0636 and Rv0636-Rv0637) correlate with their specificity for long-chain substrates C₁₂), it being possible for the noncatalytic subunit to play the role of acyl long chain acceptor; (iii) these enzymes appear to exhibit a marked specificity for ACP derivatives compared with CoA derivatives; (iv) their chain-length specificity and their specificity for the acyl-chain carrier group of the substrate correspond to the properties described for the mycobacterial FAS-II system and for the enzymes of which it is composed; (v) the preference of Rv0636-Rv0637 for longer-chain substrates compared with Rv0635-Rv0636, and the absence of a protein orthologous to Rv0637 in the Mycobacterium-related genera comprising short mycolic acids, suggest that Rv0635-Rv0636 is involved in the early elongation cycles catalyzed by FAS-II, whereas Rv0636-Rv0637 is involved in the late elongation cycles.

### LITERATURE REFERENCES

Altschul, S. F., Madden, T. L., Schaffer, A. A., Zhang, J., Zhang, Z., Miller,

W. & Liprnan, D. J. (1997). Gapped BLAST and PSI-BLAST: a new generation of protein database search programs. Nucleic Acids Res 25, 3389-3402.
Aranaz, A., Cousins, D., Mateos, A. & Dominguez, L. (2003). Elevation of Mycobacterium tuberculosis subsp. caprae Aranaz et al. 1999 to species rank as Mycobacterium caprae comb. nov., sp. nov. Int J Syst Evol Microbiol 53, 1785-1789.
Banerjee, A., Sugantino, M., Sacchettini, J. C. & Jacobs, W. R., Jr. (1998). The mabA gene from the inhA operon of Mycobacterium tuberculosis encodes a 3-ketoacyl reductase that fails to confer isoniazid resistance. Microbiology 144 (Pt 10), 2697-2704.
Barry, C. E., 3rd, Lee, R. E., Mdluli, K., Sampson, A. E., Schroeder, B. G., Slayden, R. A. & Yuan, Y. (1998). Mycolic acids: structure, biosynthesis and physiological functions. Prog Lipid Res 37, 143-179.
Besra, G. S., Minnikin, D. E., Wheeler, P. R. & Ratledge, C. (1993). Synthesis of methyl (Z)-tetracos-5-enoate and both enantiomers of ethyl (E)-6-methyl-tetracos-4-enoate: possible intermediates in the biosynthesis of mycolic acids in mycobacteria. Chem Phys Lipids 66, 23-34.
Brennan, P. J. & Nikaido, H. (1995). The envelope of mycobacteria. Annu. Rev Biochem 64, 29-63.
Brown, A. K., Papaemmanouil, A., Bhowruth, V., Bhatt, A., Dover, L. G., Besra, G. S. (2007). Flavonoid inhibitors as novel antimycobacterial agents targeting Rv0636, a putative dehydratase enzyme involved in Mycobacterium tuberculosis fatty acid synthase II. Microbiology 153, 3314-22.
Choi, K. H., Kremer, L., Besra, G. S. & Rock, C. O. (2000). Identification and substrate specificity of beta-ketoacyl (acyl earner protein) synthase III (mtFabH) from Mycobacterium tuberculosis. J Biol Chem 275, 28201-28207.
Cole, S. T., Brosch, R., Parkhill, J. & other authors (1998). Deciphering the biology of Mycobacterium tuberculosis from the complete genome sequence. Nature 393, 537 -544.
Corpet, F. (1998). Multiple sequence alignment with hierarchical clustering. Nucleic Acids Res 16, 10881-10890.
Cousins, D. V., Bastida, R., Cataldi, A. & other authors (2003). Tuberculosis in seals caused by a novel member of the Mycobacterium tuberculosis complex: Mycobacterium pinnipedii sp. nov. Int J Syst Evol Microbiol 53, 1305-1314.
Cronan, J. E. J. & Klages, A. L. (1981). Chemical synthesis of acyl thioesters of acyl carrier protein with native structure. Proc Natl Acad Sci U S A 78, 5440-5444.
Fernandes, N. D. & Kolattukudy, P. E. (1996). Cloning, sequencing and characterization of a fatty acid synthase-encoding gene from Mycobacterium tuberculosis var. bovis BCG. Gene 170, 95-99.
Gattilter, A., Gasteiger, E. & Bairoch, A. (2002). ScanProsite: a reference implementation of a PROSITE scanning tool. Appl Bioinformatics 1, 107-108.
Goldman, P. & Vagelos, P. R. (1961). The specificity of triglyceride synthesis from diglycerides in chicken adipose tissue. J Biol Chem 236, 2620-2623.
Hisano, T., Tsuge, T., Fukui, T., Iwata, T., Miki, K., Doi, Y. (2003). Crystal structure of the (R)-specific enoyl-CoA hydratase from Aeromonas caviae involved in polyhydroxyalkanoate biosynthesis. J Biol Chem. 278, 617-24.
Jonassen, I., Collins, J. F. & Higgins, D. (1995). Finding flexible patterns in unaligned protein sequences. Protein Sci 4, 1587-1595.
Kastaniotis, A. J., Autio, K. J., Sormunen, R. T. & Hiltunen, J. K. (2004). Htd2p/Yhr067p is a yeast 3-hydroxyacyl-ACP dehydratase essential for mitochondrial function and morphology. Mol Microbiol 53, 1407-1421.
Kremer, L., Nampoothiri, K. M., Lesjean, S. & other authors (2001). Biochemical characterization of acyl carrier protein (AcpM) and malonyl-CoA:AcpM transacylase (mtFabD), two major components of Mycobacterium tuberculosis fatty acid synthase II. J Biol Chem 276, 27967-27974.
Kremer, L., Dover, L. G., Carrere, S. & other authors (2002). Mycolic acid biosynthesis and enzymic characterization of the beta-kctoacyl-ACP synthase A-condensing enzyme from Mycobacterium tuberculosis. Biochem J 364, 423-430.
Leeson, M., Henderson, B. S. Gillig, J. R., Schwab, J. M., Smith, J. L. (1996). Structure of a dehydratase-isomerase from the bacterial pathway for biosynthesis of unsaturated fatty acids: two catalytic activities in one active site. Structure IS, 253-64.
Marrakchi, H., Lancelile, G. & Quemard, A. (2000). InhA, a target of the antituberculous drug isoniazid, is involved in a mycobacterial fatty acid elongation system, FAS-II. Microbiology 146 (Pt 2), 289-296.
Marrakchi, H., Choi, K. H. & Rock, C. O. (2002a). A new mechanism for anaerobic unsaturated fatty acid formation in Streptococcus pneumoniae. J Biol Chem 277, 44809-44816.
Marrakchi, H., Ducasse, S., Labcsse, G., Montrozier, H., Margeat, E., Emorine, L., Charpentier, X., Daffe, M. & Quemard, A. (2002b). MabA (FabG1), a Mycobacterium tuberculosis protein involved in the long-chain fatty acid elongation system FAS-II. Microbiology 148, 951-960.
Quémard, A., Sacchettini, J. C., Dessen, A., Vilcheze, C., Bittman, R., Jacobs, W. R., Jr. & Blanchard, J. S. (1995). Enzymatic characterization of the target for isoniazid in Mycobacterium tuberculosis. Biochemistry 34, 8235-8241.
Ratledge, C. (1982). Lipids: cell composition, fatty acid biosyntheses. In The biology of the mycobacteria, pp. 53-93. Edited by C. S. Ratledge, J. London: Academic Press Inc.
Schaeffer, M. L., Agnihotri, G., Kallender, H., Brennan, P. J. & Lonsdale, J. T. (2001a). Expression, purification, and characterization of the Mycobacterium tuberculosis acyl carrier protein, AcpM. Biochim Biophys Acta 1532, 67-78.
Schaeffer, M. L., Agnihotri, G., Volker, C., Kallender, H., Brennan, P. J. & Lonsdale, J. T. (2001b). Purification and biochemical characterization of the Mycobacterium tuberculosis beta-ketoacyl-acyl carrier protein synthases KasA and KasB. J Biol Chem 276, 47029-47037.
Thompson, J. D., Higgins, D. G. & Gibson, T. J. (1994). Clustal W: improving the sensitivity of progressive multiple sequence alignment through sequence weighting, position specific gap penalties and weight matrix choice. Nucleic Acids Res 22, 4673-4680.
van Soolingen, D., Hoogenboezem, T., de Haas, P. E. & other authors (1997). A novel pathogenic taxon of the Mycobacterium tuberculosis complex, Canetti: characterization of an exceptional isolate from Africa. Int J Syst Bacteriol 47, 1236-1245.
Wang, H. & Cronan, J. E. (2004). Functional replacement of the FabA and FabB proteins of Escherichia coli fatty acid synthesis by Enterococcus faecalis FabZ and FabF homologues. J Biol Chem 279, 34489-34495.

1. A purified and isolated enzyme, involved in the FAS II system and having the following characteristics:
a) it is constituted of a dimer or a multimer selected from the group consisting of:
(i) homomultimers comprising at least three identical proteins comprising a hydratase 2 motif [YF]-x(1,2)-[LIVG]-[STGCFG-D-x-N-P-[LIV]-H-x(5)-[AS] (SEQ ID No. 15), (which x(n) represents any n amino acids, and the amino acids between square brackets representing alternatives), or a catalytic motif derived from the hydratase 2 motif and constituted at least of the basic motif D-x(4) H, in which x(4) represents any 4 amino acids, or a motif similar to the basic motif,
(ii) heterodimers and heteromultimers comprising:
at least two different proteins as defined in (i), or
one or more proteins as defined in (i) and of one or more proteins not comprising a hydratase 2 motif, but the three-dimensional structure of which exhibits a hotdog fold; and
b) it catalyzes at least one of the dehydration of a (3R)-hydroxyacyl substrate to give 2 trans-enoyl and the hydration of a 2 trans-enoyl substrate to give (3R)-hydroxyacyl, in accordance with scheme (I): 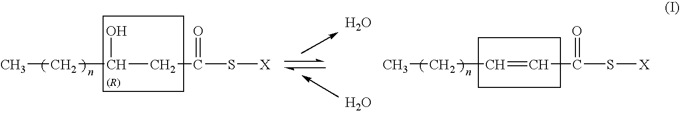
in which X represents ACP and n≧0, or CoA and n≧0.

2. The purified and isolated enzyme as claimed in claim 1, wherein, when X represents CoA, then n≧8.

3. The purified and isolated enzyme as claimed in claim 1, wherein the protein as defined in (i) is selected from the group consisting of the Rv0636 protein (SEQ ID No. 2) comprising a hydratase 2 motif Y-A-G-V—S-G-D-L-N—P—I—H—W-D-D-E-I-A (SEQ ID No. 16) and a protein which has at least 51% identity or at least 72% similarity, with the Rv0636 protein of SEQ ID No. 2 of M. tuberculosis H37Rv.

4. The isolated and purified enzyme as claimed in claim 1, wherein the protein as defined in (ii), which does not comprise a hydratase 2 motif, is selected from the group consisting of:
the Rv0635 protein (SEQ ID No. 4), a protein which has at least 53% identity or at least 67% similarity, with the Rv0635 protein of sequence SEQ ID No. 4 of M. tuberculosis H37Rv,
the Rv0637 protein (SEQ ID No. 6), and a protein which has at least 44% identity or at least 61% similarity, with the Rv0637 protein of sequence SEQ ID No. 6 of M. tuberculosis H37Rv.

5. The purified and isolated enzyme as claimed in claim 1, wherein the dimer or multimer is:
a homomultimer of Rv0636 (SEQ ID No. 2) or of a protein which has at least 51% identity or at least 72% similarity, over its entire sequence, with the Rv0636 protein of M. tuberculosis,
a heterodimer or a heteromultimer constituted (i) of the Rv0636 protein (SEQ ID No. 2) or of a protein which has at least 51% identity or at least 72% similarity, with the Rv0636 protein of sequence SEQ ID No. 2 of M. tuberculosis H37Rv and (ii) of the Rv0635 protein (SEQ ID No. 4) or of a protein which has at least 53% identity or at least 67% similarity, with the Rv0635 protein of sequence SEQ ID No. 4 of M. tuberculosis H37Rv, or
a heterodimer or a heteromultimer constituted (i) of the Rv0636 protein (SEQ ID No. 2) or of a protein which has at least 51% identity or at least 72% similarity, with the Rv0636 protein of sequence SEQ ID No. 2 of M. tuberculosis H37Rv and (ii) of the Rv0637 protein (SEQ ID No. 6) or of a protein which has at least 44% identity or at least 61% similarity, with the Rv0637 protein of sequence SEQ ID No. 6 of M. tuberculosis H37Rv.

6. The purified and isolated enzyme as claimed in claim 1, wherein it is selected from the group consisting of: the homomultimer of Rv0636, the heterodimer of Rv0635-Rv0636, the heteromultimer Rv0635-Rv0636, the heterodimer of Rv0636-Rv0637, and the heteromultimer Rv0636-Rv0637.

7. The purified and isolated enzyme as claimed in claim 1, wherein at least one of the (3R)-hydroxyacyl substrate and the 2 trans-enoyl substrate for the enzyme is a substrate having an acyl chain of length greater than or equal to C₈, derived from ACP.

8. An isolated polynucleotide, which encodes an enzyme as claimed claim 1 or a protein selected from the group consisting of the Rv0635, Rv0636 and Rv0637 proteins.

9. The polynucleotide as claimed in claim 8, which is selected from the group consisting of the polynucleotide of SEQ ID No. 1, the polynucleotide of SEQ ID No. 3, the polynucleotide of SEQ ID No. 5, the polynucleotide comprising at least one of the sequence which encodes an Rv0636 protein, the sequence which encodes an Rv0635 protein, and the sequence which encodes an Rv0637 protein.

10. A polynucleotide, comprising the reading frames encoding the Rv0635, Rv0636 and Rv0637 proteins, wherein said polynuleotide represented by the sequence SEQ ID No. 7 of the sequence listing attached in the annex.

11. An expression vector, comprising a polynucleotide as claimed in claim 8.

12. The vector as claimed in claim 11, comprising a polynucleotide encoding an Rv0636 protein and at least one polynucleotide encoding the Rv0635 protein, the Rv0637 protein, or a combination thereof.

13. The vector as claimed in claim 11, further comprising the regulatory sequences for the expression of the protein.

14. A prokaryotic or eukaryotic host cell, transformed with the expression vector as claimed in claim 11.

15. A method for producing said purified and isolated enzyme or a protein selected from the group consisting of the Rv0635, Rv0636 and Rv0637 proteins, comprising culturing a host cell as claimed in claim 14 in a medium suitable for the purification of the enzyme or of the protein from said culture.

16. The method as claimed in claim 15, wherein said purification is carried out by affinity chromatography, by exclusion chromatography, or by a combination thereof.

17. A method for inhibiting the biosynthesis of compounds of the envelope of mycolata, comprising inhibiting the expression or the activity of at least one of the enzymes as claimed in claim 1, or of a protein selected from the group consisting of the Rv0636 protein, the Rv0635 protein and the Rv0637 protein.

18. A method for inhibiting the biosynthesis of compounds of the envelope of mycolata, comprising:
a) incubating a substrate selected from the group constituted of (3R)-hydroxyacyl-ACPs, trans-2 enoyl-ACPs, (3R)-hydroxyacyl-CoAs and trans-2 enoyl-CoAs, with an enzyme as claimed in claim 1, in the presence or absence of the test molecule, and
b) comparing the activity of said enzyme in the presence and absence of the test molecule.

19. The method as claimed in claim 18, wherein said test molecule inhibits the biosynthesis of compounds of the envelope of pathogenic mycobacteria and of other pathogenic bacteria among the Corynebacterinae.

20-23. (canceled)

24. A method for screening for ligands of an enzyme as claimed in claim 1 or of a protein selected from the group constituted of the RV0635, Rv0636 and Rv0637 proteins, comprising:
contacting said enzyme or a protein selected from the group constituted of the RV0635, Rv0636 and Rv0637 proteins with a reaction medium comprising the potential ligand to be tested,
after excitation at 303 nm, measuring the variation in fluorescence intensity at the maximum emission,
and on the basis of this measurement, detecting the binding of said ligand to one or more of the proteins constituting said enzyme.

25. The screening method as claimed in claim 24, wherein the enzyme is the Rv0636 protein or an enzyme containing same.

26. A method for screening for ligands of an enzyme as claimed in claim 1 or of a protein selected from the group consisting of the Rv0635, Rv0636 and Rv0637 proteins, comprising:
contacting said enzyme or a protein selected from the group constituted of the Rv0635, Rv0636 and Rv0637 proteins with the ligand to be tested, and
analyzing the complex formed in the soluble phase between said enzyme and said ligand, by NMR, by fluorescence, or by a combination thereof.

27. A method for screening for ligands which inhibit an enzyme as claimed in claim 1 or a protein selected from the group consisting of the Rv0635, Rv0636 and Rv0637 proteins, comprising identifying the inhibition of the expression or of the activity of said enzyme or of said protein.

28. The purified and isolated enzyme according to claim 1, wherein n in scheme (I) is ≧4.

29. The purified and isolated enzyme as claimed in claim 1, wherein the protein as defined in (i) is selected from the group consisting of the Rv0636 protein (SEQ ID No. 2) comprising a hydratase 2 motif Y-A-G-V-S-G-D-L-N—P—I—H—W-D-D-E-I-A (SEQ ID No. 16) and a protein which has at least 85% identity or at least 90% similarity with the Rv0636 protein of SEQ ID No. 2 of M. tuberculosis H37Rv.

30. The isolated and purified enzyme as claimed in claim 1, wherein the protein as defined in (ii), which does not comprise a hydratase 2 motif, is selected from the group consisting of:
the Rv0635 protein (SEQ ID No. 4), a protein which has at least 69% identity or at least 82% similarity with the Rv0635 protein of sequence SEQ ID No. 4 of M. tuberculosis H37Rv,
the Rv0637 protein (SEQ ID No. 6), and a protein which has at least 59% identity or at least 76% similarity with the Rv0637 protein of sequence SEQ ID No. 6 of M. tuberculosis H37Rv.

31. The purified and isolated enzyme as claimed in claim 1, wherein the dimer or multimer is:
a homomultimer of Rv0636 (SEQ ID No. 2) or of a protein which has at least 85% identity or at least 90% similarity over its entire sequence, with the Rv0636 protein of M. tuberculosis,
a heterodimer or a heteromultimer constituted (i) of the Rv0636 protein (SEQ ID No. 2) or of a protein which has at least 85% identity or at least 90% similarity with the Rv0636 protein of sequence SEQ ID No. 2 of M. tuberculosis H37Rv and (ii) of the Rv0635 protein (SEQ ID No. 4) or of a protein which has at least 69% identity or at least 82% similarity with the Rv0635 protein of sequence SEQ ID No. 4 of M. tuberculosis H37Rv, or
a heterodimer or a heteromultimer constituted (i) of the Rv0636 protein (SEQ ID No. 2) or of a protein which has at least 85% identity or at least 90% similarity with the Rv0636 protein of sequence SEQ ID No. 2 of M. tuberculosis H37Rv and (ii) of the Rv0637 protein (SEQ ID No. 6) or of a protein which has at least 59% identity or at least 76% similarity with the Rv0637 protein of sequence SEQ ID No. 6 of M. tuberculosis H37Rv.

32. The purified and isolated enzyme as claimed in claim 1, wherein at least one of the (3R)-hydroxyacyl substrate and the 2 trans-enoyl substrate for the enzyme is a substrate having an acyl chain of length C₁₂-C₂₀, derived from ACP.

33. A prokaryotic or eukaryotic host cell, transformed with a polynucleotide as claimed in claim 8.

34. The method as claimed in claim 15, wherein said purification is carried out by IMAC.
